(19) United States
(12) Patent Application Publication
(10) Pub. No.: US 2026/0253122 A1
(43) Pub. Date: Aug. 27, 2026

(54) SYSTEM

(71) Applicant: SoftBank Group Corp., Tokyo (JP)

(72) Inventor: Ryunosuke AMANO, Tokyo (JP)

(73) Assignee: SoftBank Group Corp., Tokyo (JP)

(21) Appl. No.: 19/541,452

(22) Filed: Feb. 17, 2026

(30) Foreign Application Priority Data
Feb. 21, 2025 (JP) 2025-026976

(51) Int. Cl. G06Q 30/0601

(52) U.S. Cl. CPC G06Q 30/0631

(57) ABSTRACT

The system according to the embodiment comprises a collection unit, an analysis unit, a proposal unit, an ordering unit, and a recommendation unit. The collection unit collects health and exercise information. The analysis unit analyzes information collected by the collection unit. The proposal unit proposes cooking recipes based on analysis results obtained by the analysis unit. The ordering unit places orders for ingredients based on recipes proposed by the proposal unit. The recommendation unit provides dining-out information based on recipes proposed by the proposal unit.

### CROSS-REFERENCE TO RELATED APPLICATION

The present application claims priority to and incorporates by reference the entire contents of Japanese Patent Application No. 2025-026976 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, optimal proposals of cooking recipes, ordering of ingredients, and provision of dining-out information based on individual health conditions and exercise information have not been sufficiently performed, and there is room for improvement.

### SUMMARY OF THE INVENTION

The system according to the embodiment comprises a collection unit, an analysis unit, a proposal unit, an ordering unit, and a recommendation unit. The collection unit collects health and exercise information. The analysis unit analyzes information collected by the collection unit. The proposal unit proposes cooking recipes based on analysis results obtained by the analysis unit. The ordering unit places orders for ingredients based on recipes proposed by the proposal unit. The recommendation unit provides dining-out information based on recipes proposed by the proposal unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

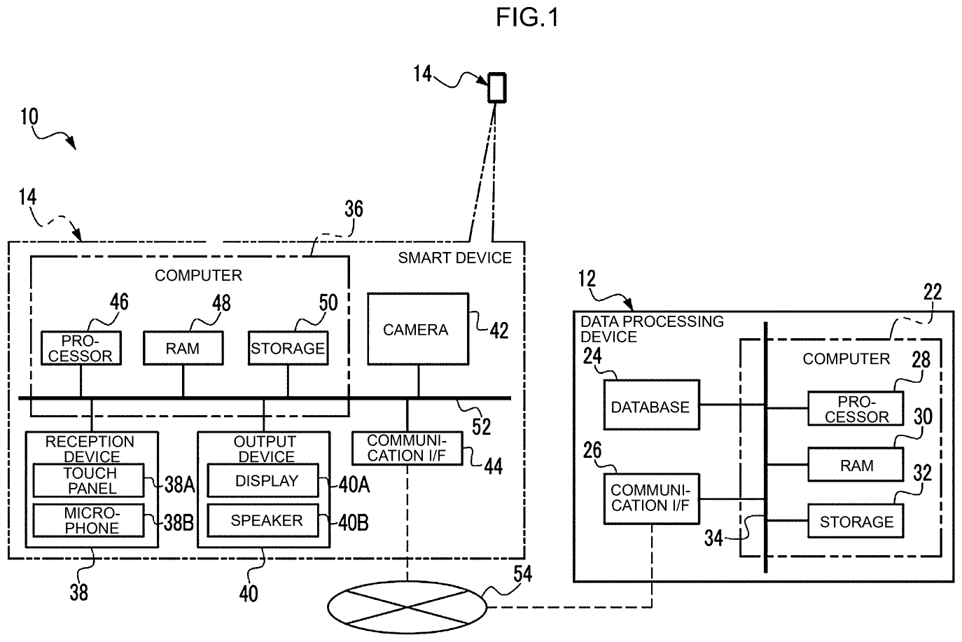

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

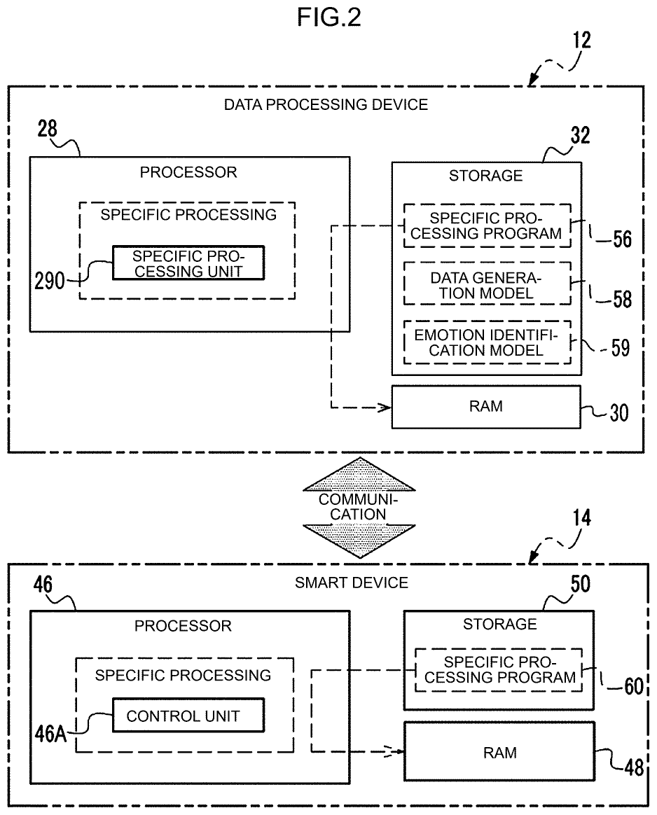

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system that proposes optimal cooking recipes for a person at a given timing, based on various health and exercise information automatically acquired from wearable devices, health checkup results, daily meal photographs, and other inputs. This system collects and analyzes health and exercise information, proposes optimal cooking recipes, and enables ordering of ingredients and provision of dining-out information. For example, data such as heart rate, step count, calories burned, and sleep duration are collected from wearable devices, thereby allowing the system to grasp the person's health conditions and exercise status. Next, data such as health checkup results and daily meal photographs are collected, including blood pressure, blood glucose level, cholesterol level, and meal photographs, which enable the system to grasp the person's health conditions and dietary trends. The collected data are then analyzed. A generative AI analyzes the collected data to grasp the person's health conditions, exercise status, and dietary trends. For example, the AI analyzes data such as heart rate, step count, and calories burned to determine exercise status, and analyzes health checkup results and meal photographs to determine health conditions and dietary trends. Based on these analyses, the system can propose optimal cooking recipes for the person. Furthermore, based on the analysis results, the generative AI proposes cooking recipes tailored to the person's health conditions, exercise status, and dietary trends. For example, low-calorie cooking recipes are proposed for people on a diet, nutritionally balanced recipes for people undergoing medical treatment, and high-protein recipes for professional athletes. Thus, the system can propose optimal cooking recipes for each individual. Based on the proposed recipes, orders for ingredients can be placed with nearby online supermarkets. The generative AI automatically selects necessary ingredients based on the proposed recipes and places orders with nearby online supermarkets, allowing users to obtain necessary ingredients without hassle. For those who are not good at cooking at home, the system selects and recommends dining-out information with menus similar to the relevant recipes. The generative AI searches menus of nearby restaurants and cafes based on the proposed recipes, selects dining-out information with menus similar to the relevant recipes, and provides recommendations, enabling even those who are not good at cooking at home to enjoy healthy meals. Thus, the system can propose optimal cooking recipes and provide ingredient ordering and dining-out information based on the user's health conditions, exercise status, and dietary trends. Specifically, the system receives as input biometric information obtained from wearable devices (e.g., heart rate as a time-series array at one-minute intervals, step count as a daily integer, calories burned as a daily real value, sleep duration as a daily real value), health checkup results (e.g., blood pressure as two-dimensional values for systolic and diastolic, blood glucose level as a real value in mg/dL, cholesterol level as a real value in mg/dL), and meal photographs (e.g., RGB image data, resolution 640×480 pixels, JPEG format). The system performs preprocessing such as normalization, missing value imputation, time-series alignment, and image feature extraction (image encoding by CNN) on these diverse data in a preprocessing unit, and integrates them into unified feature vectors (e.g., biometric information as a 10-dimensional vector, image features as a 512-dimensional vector). The integrated feature vectors are input to a Transformer-based multilayer neural network, which simultaneously infers multiple tasks such as health condition classification (e.g., healthy, caution, needs improvement), exercise status estimation (e.g., active, standard, low activity), and dietary trend clustering (e.g., high-fat trend, low-carb trend, vegetable deficiency trend). For example, input examples include “heart rate: 72 bpm, step count: 8,000 steps, calories burned: 2,100 kcal, sleep duration: 7.5 hours, blood pressure: 120/80 mmHg, blood glucose: 95 mg/dL, meal photo: image of vegetable salad and chicken” or “heart rate: 90 bpm, step count: 3,000 steps, calories burned: 1,500 kcal, sleep duration: 5 hours, blood pressure: 140/90 mmHg, blood glucose: 130 mg/dL, meal photo: image of hamburger and fries.” From these inputs, the system generates structured output data such as “health condition: needs improvement, recommended recipe: low-calorie high-protein salad, ingredients: chicken breast, lettuce, tomato, olive oil, order destination: nearby online supermarket A, dining-out candidate: grilled chicken salad at cafe B” or “health condition: healthy, recommended recipe: balanced Japanese meal, ingredients: salmon, spinach, brown rice, order destination: online supermarket C, dining-out candidate: set meal at Japanese restaurant D.” The system branches the inference results in a threshold judgment unit according to rules such as “prioritize low-calorie recipes only when health condition is needs improvement,” and distributes data to the proposal unit, ordering unit, and recommendation unit. During neural network training, the system uses a weighted sum of health condition classification error, recipe proposal accuracy, and ingredient selection accuracy as the loss function, and optimizes weights by gradient descent. Data augmentation such as rotation and brightness adjustment of meal photographs and noise addition to health checkup values is performed to improve generalization performance. Unlike conventional human-based simple health management and recipe selection, the system implements computer-specific non-conventional procedures such as multivariate analysis in high-dimensional feature space, rule-based branching, automatic ingredient selection, and dining-out candidate recommendation, thereby achieving significant improvements in processing speed, proposal accuracy, reduction of user burden, and efficiency of data management. Specific application fields include personal health management applications, corporate health management support systems, nutrition management services for athletes, and dietary guidance support systems for medical institutions.

The health management system according to the embodiment comprises a collection unit, an analysis unit, a proposal unit, an ordering unit, and a recommendation unit. The collection unit collects health and exercise information. Health and exercise information may include, for example, heart rate, step count, calories burned, and meal content, but is not limited thereto. The collection unit automatically collects data such as heart rate, step count, calories burned, and sleep duration from wearable devices. The collection unit can also collect data such as health checkup results and daily meal photographs, including blood pressure, blood glucose level, cholesterol level, and meal photographs. The analysis unit analyzes information collected by the collection unit. For example, the analysis unit analyzes the collected data using statistical analysis or machine learning algorithms to grasp the user's health conditions, exercise status, and dietary trends. The proposal unit proposes cooking recipes based on analysis results obtained by the analysis unit. For example, the proposal unit proposes optimal cooking recipes based on the user's health conditions, exercise status, and dietary trends. For example, low-calorie cooking recipes are proposed for people on a diet, nutritionally balanced recipes for people undergoing medical treatment, and high-protein recipes for professional athletes. The ordering unit places orders for ingredients based on recipes proposed by the proposal unit. For example, the ordering unit automatically selects necessary ingredients based on the proposed cooking recipes and places orders with nearby online supermarkets. The recommendation unit provides dining-out information based on recipes proposed by the proposal unit. For example, the recommendation unit searches menus of nearby restaurants and cafes based on the proposed cooking recipes, selects dining-out information with menus similar to the relevant recipes, and provides recommendations. Thus, the health management system according to the embodiment can propose optimal cooking recipes and provide ingredient ordering and dining-out information based on the user's health conditions, exercise status, and dietary trends. Specifically, the health management system receives as input biometric information obtained from wearable devices (e.g., heart rate as a time-series array at one-minute intervals, step count as a daily integer, calories burned as a daily real value, sleep duration as a daily real value), health checkup results (e.g., blood pressure as two-dimensional values for systolic and diastolic, blood glucose level as a real value in mg/dL, cholesterol level as a real value in mg/dL), and meal photographs (e.g., RGB image data, resolution 640×480 pixels, JPEG format). The system performs preprocessing such as normalization, missing value imputation, time-series alignment, and image feature extraction (image encoding by convolutional neural network) in a preprocessing unit, and integrates the data into unified feature vectors (e.g., biometric information as a 10-dimensional vector, image features as a 512-dimensional vector). The integrated feature vectors are input to a Transformer-based multilayer neural network, which simultaneously infers multiple tasks such as health condition classification (e.g., healthy, caution, needs improvement), exercise status estimation (e.g., active, standard, low activity), and dietary trend clustering (e.g., high-fat trend, low-carb trend, vegetable deficiency trend). For example, input examples include “heart rate: 72 bpm, step count: 8,000 steps, calories burned: 2,100 kcal, sleep duration: 7.5 hours, blood pressure: 120/80 mmHg, blood glucose: 95 mg/dL, meal photo: image of vegetable salad and chicken” or “heart rate: 90 bpm, step count: 3,000 steps, calories burned: 1,500 kcal, sleep duration: 5 hours, blood pressure: 140/90 mmHg, blood glucose: 130 mg/dL, meal photo: image of hamburger and fries.” From these inputs, the system generates structured output data such as “health condition: needs improvement, recommended recipe: low-calorie high-protein salad, ingredients: chicken breast, lettuce, tomato, olive oil, order destination: nearby online supermarket A, dining-out candidate: grilled chicken salad at cafe B” or “health condition: healthy, recommended recipe: balanced Japanese meal, ingredients: salmon, spinach, brown rice, order destination: online supermarket C, dining-out candidate: set meal at Japanese restaurant D.” The system branches the inference results in a threshold judgment unit according to rules such as “prioritize low-calorie recipes only when health condition is needs improvement,” and distributes data to the proposal unit, ordering unit, and recommendation unit. During neural network training, the system uses a weighted sum of health condition classification error, recipe proposal accuracy, and ingredient selection accuracy as the loss function, and optimizes weights by gradient descent. Data augmentation such as rotation and brightness adjustment of meal photographs and noise addition to health checkup values is performed to improve generalization performance. Unlike conventional human-based simple health management and recipe selection, the system implements computer-specific non-conventional procedures such as multivariate analysis in high-dimensional feature space, rule-based branching, automatic ingredient selection, and dining-out candidate recommendation, thereby achieving significant improvements in processing speed, proposal accuracy, reduction of user burden, and efficiency of data management. Specific application fields include personal health management applications, corporate health management support systems, nutrition management services for athletes, and dietary guidance support systems for medical institutions.

The collection unit can collect health checkup results or daily meal photographs. For example, the collection unit collects health checkup results, which may include blood test results, electrocardiogram results, and the like, but are not limited thereto. The collection unit can also collect daily meal photographs, which may include photographs of entire meals or specific dishes, but are not limited thereto. By collecting health checkup results and meal photographs, the collection unit can obtain more detailed health and exercise information. Specifically, the collection unit acquires digital data such as blood test results (e.g., numerical vectors for red blood cell count, white blood cell count, hemoglobin value), electrocardiogram results (e.g., 12-lead ECG waveform data, time-series arrays at one-second intervals, float value arrays of 500 samples per lead), and meal photographs as RGB image data (e.g., resolution 640×480 pixels, JPEG format), managing multiple images per day in a time-series manner. When acquiring these data, the collection unit automatically performs preprocessing such as missing value imputation, anomaly detection (e.g., exclusion of physiologically impossible values), image resizing, and noise removal. The collection unit links health checkup results and meal photographs and stores them in a time-series database, enabling tracking of changes in health conditions and dietary trends for each user. Thus, the collection unit achieves a technical effect of significantly improving data comprehensiveness, accuracy, and time-series consistency by integrally and automatically collecting and managing multiple sensor data and image data, rather than simple manual recording by humans. Specific application fields include remote health monitoring services, dietary guidance applications, and patient management systems for medical institutions.

The analysis unit analyzes the collected data to grasp health conditions, exercise status, and dietary trends. For example, the analysis unit analyzes the collected data using statistical analysis or machine learning algorithms. Health conditions may include, for example, body weight, blood pressure, blood glucose level, and the like, but are not limited thereto. Exercise status may include, for example, exercise frequency, exercise type, exercise intensity, and the like, but are not limited thereto. Dietary trends may include, for example, calorie intake, nutritional balance, and the like, but are not limited thereto. By analyzing the collected data, the analysis unit can grasp the user's health conditions, exercise status, and dietary trends. Specifically, the analysis unit receives as input health checkup data (e.g., numerical vectors for blood pressure, blood glucose level, body weight) from the collection unit, exercise data (e.g., time-series arrays for step count, calories burned, exercise intensity), and dietary data (e.g., image feature vectors of meal photographs, estimated calorie values). The analysis unit first performs preprocessing such as normalization, outlier removal, and time-series alignment to generate unified feature vectors. For health condition estimation, machine learning models such as random forest or multilayer perceptron are used to classify health conditions (e.g., healthy, caution, needs improvement) from input features. For exercise status estimation, recurrent neural networks are used to extract patterns of exercise frequency and intensity from time-series data. For dietary trend analysis, image feature extraction (encoding by convolutional neural network) and nutrient estimation algorithms are combined to estimate calorie intake and nutritional balance. For example, input examples include “blood pressure: 130/85 mmHg, blood glucose: 110 mg/dL, step count: 6,000 steps, meal photo: image of pasta and salad,” and output examples include “health condition: caution, exercise status: standard, dietary trend: high carbohydrate.” The analysis unit branches inference results in a threshold judgment unit, such as sending detailed dietary guidance to subsequent processing only when health condition is needs improvement. Thus, the analysis unit achieves a technical effect of significantly improving analysis accuracy, speed, and consistency by realizing multivariate analysis in high-dimensional feature space, automatic classification and pattern extraction by machine learning models, unlike conventional simple statistical aggregation or human visual judgment. Specific application fields include automatic analysis services for health checkup results, exercise guidance applications, and dietary trend analysis tools.

The proposal unit can propose low-calorie cooking recipes for people on a diet. For example, the proposal unit proposes low-calorie cooking recipes for people on a diet, where low-calorie means, for example, a small amount of calories per meal, but is not limited thereto. By proposing low-calorie cooking recipes for people on a diet, the proposal unit can support healthy dieting. Specifically, the proposal unit receives as input feature vectors such as the user's health condition (e.g., needs improvement), exercise status (e.g., low activity), and dietary trend (e.g., high-fat trend) from the analysis unit. The proposal unit implements a calorie restriction algorithm (e.g., prioritizing recipes with less than 500 kcal per meal), searching and scoring cooking recipes in the recipe database that meet the relevant conditions. For example, input examples include “health condition: needs improvement, goal: diet, current calorie intake: 2,200 kcal/day,” and output examples include “recommended recipes: steamed chicken breast and vegetables (350 kcal per meal), tofu salad (180 kcal per meal).” The proposal unit outputs structured data for each recommended recipe, including ingredient lists, cooking procedures, and nutrient information, and links to subsequent ordering and recommendation units. Thus, the proposal unit achieves a technical effect of significantly improving the effectiveness, convenience, and continuity of diet support by realizing automatic and highly accurate recipe proposals based on individual health conditions, goals, and dietary trends, unlike conventional human experience or visual selection. Specific application fields include diet support applications, nutrition guidance services, and corporate health management support systems.

The proposal unit can propose nutritionally balanced cooking recipes for people undergoing medical treatment. For example, the proposal unit proposes nutritionally balanced cooking recipes for people undergoing medical treatment, where nutritionally balanced means, for example, a good balance of protein, fat, and carbohydrates, but is not limited thereto. By proposing nutritionally balanced cooking recipes for people undergoing medical treatment, the proposal unit can support healthy eating. Specifically, the proposal unit receives as input feature vectors such as health condition (e.g., caution, needs improvement) and dietary trend (e.g., vegetable deficiency trend, excessive fat trend) from the analysis unit. The proposal unit implements a nutritional balance algorithm (e.g., prioritizing recipes with PFC balance close to recommended ratios), referencing nutrient information such as protein, fat, carbohydrates, vitamins, and minerals in the recipe database to search and score optimal cooking recipes. For example, input examples include “health condition: caution, goal: medical treatment, current PFC ratio: P10% F40% C50%,” and output examples include “recommended recipes: salmon foil bake (P25% F20% C55%), spinach ohitashi (P20% F10% C70%).” The proposal unit outputs structured data for each recommended recipe, including ingredient lists, cooking procedures, and nutrient information, and links to subsequent ordering and recommendation units. Thus, the proposal unit achieves a technical effect of significantly improving the effectiveness, convenience, and continuity of medical treatment support by realizing automatic and highly accurate recipe proposals based on individual health conditions, nutritional balance, and dietary trends, unlike conventional human experience or visual selection. Specific application fields include medical diet support applications, dietary guidance systems for medical institutions, and nutrition management services.

The proposal unit can propose high-protein cooking recipes for professional athletes. For example, the proposal unit proposes high-protein cooking recipes for professional athletes, where high-protein means, for example, a large amount of protein per meal, but is not limited thereto. By proposing high-protein cooking recipes for professional athletes, the proposal unit can maximize the effectiveness of training. Specifically, the proposal unit receives as input feature vectors such as exercise status (e.g., high-intensity training), health condition (e.g., healthy), and dietary trend (e.g., low-protein trend) from the analysis unit. The proposal unit implements a high-protein algorithm (e.g., prioritizing recipes with 30 g or more protein per meal), searching and scoring cooking recipes in the recipe database that meet the relevant conditions. For example, input examples include “exercise status: high intensity, goal: muscle strengthening, current protein intake: 70 g/day,” and output examples include “recommended recipes: grilled chicken and bean salad (protein 35 g), salmon steak (protein 32 g).” The proposal unit outputs structured data for each recommended recipe, including ingredient lists, cooking procedures, and nutrient information, and links to subsequent ordering and recommendation units. Thus, the proposal unit achieves a technical effect of significantly improving athlete performance, nutrition management optimization, and continuous training support by realizing automatic and highly accurate recipe proposals based on individual exercise status, health conditions, and dietary trends, unlike conventional human experience or visual selection. Specific application fields include nutrition management applications for athletes, training support systems, and meal management services for professional teams.

The ordering unit can automatically select necessary ingredients based on the proposed cooking recipes and place orders with nearby online supermarkets. For example, the ordering unit automatically selects necessary ingredients based on the proposed cooking recipes and places orders with nearby online supermarkets, where automatic selection means selecting ingredients based on criteria such as user preferences and nutritional balance, but is not limited thereto. By automatically selecting and ordering ingredients based on the proposed cooking recipes, the ordering unit can reduce user effort. Specifically, the ordering unit receives as input the ingredient list of recommended recipes (e.g., chicken breast, lettuce, tomato, olive oil as structured data) from the proposal unit. The ordering unit refers to parameters such as the user's past purchase history, allergy information, inventory status, and preferences (e.g., prioritizing organic vegetables, specifying certain brands), and applies ingredient selection algorithms (e.g., only ordering ingredients not in stock, excluding allergenic components). For each selected ingredient, the ordering unit automatically searches for the optimal online supermarket (e.g., considering price, delivery time, inventory status), generates and transmits order data (e.g., product ID, quantity, delivery address) via API integration. For example, input examples include “recommended recipe: grilled chicken salad, ingredients: chicken breast, lettuce, tomato, olive oil, inventory: chicken breast x, lettuce ○, tomato x, olive oil ○,” and output examples include “order details: 2 chicken breasts, 3 tomatoes, order destination: online supermarket A.” After order completion, the ordering unit records the order history in a database and uses it to improve ingredient selection accuracy for future orders. Thus, the ordering unit achieves a technical effect of significantly improving order processing efficiency, preventing erroneous orders, and reducing user burden by realizing automatic and optimal ingredient selection and ordering based on individual conditions, inventory, and preferences, unlike conventional manual ordering or simple list selection by humans. Specific application fields include health management applications linked to online supermarkets, ingredient delivery services, and corporate welfare support systems.

The recommendation unit can search menus of nearby restaurants and cafes based on the proposed cooking recipes, select dining-out information with menus similar to the relevant recipes, and provide recommendations. For example, the recommendation unit searches menus of nearby restaurants and cafes based on the proposed cooking recipes, selects dining-out information with menus similar to the relevant recipes, and provides recommendations, where nearby restaurants and cafes mean those close to the user's current location, but are not limited thereto. By providing dining-out information based on the proposed cooking recipes, the recommendation unit enables even those who are not good at cooking at home to enjoy healthy meals. Specifically, the recommendation unit receives as input feature vectors of recommended recipes (e.g., dish category, main ingredients, nutrient information) from the proposal unit. The recommendation unit refers to parameters such as the user's current location (e.g., GPS coordinates), past dining-out history, and preferences (e.g., prioritizing Japanese cuisine, vegetarian options), and applies dining-out information search algorithms (e.g., calculating similarity between recipe features and restaurant menus, filtering by distance, congestion, price range). The recommendation unit scores menus similar to the recommended recipes from the menu information database of nearby restaurants and cafes and selects top candidates. For example, input examples include “recommended recipe: grilled chicken salad, current location: Chiyoda-ku, Tokyo, preference: prioritizing low-calorie,” and output examples include “dining-out candidates: grilled chicken salad at cafe B (5-minute walk), steamed chicken salad at restaurant C (8-minute walk).” The recommendation unit outputs the selected dining-out information as structured data and presents it to the user along with maps, menu details, and congestion status. Thus, the recommendation unit achieves a technical effect of significantly improving support for healthy dining-out choices, convenience, and continuity of meal management by realizing automatic and highly accurate dining-out information recommendations based on individual health conditions, preferences, and current location, unlike conventional manual search or simple list presentation by humans. Specific application fields include health-oriented dining-out recommendation applications, meal management support services, and corporate health management support systems.

The collection unit can estimate a user's emotions and adjust the timing of collecting health and exercise information based on the estimated emotions. For example, if the user is feeling stressed, the collection unit collects health and exercise information during relaxed periods. If the user is relaxed, the collection unit can also collect health and exercise information after exercise. Furthermore, if the user is in a hurry, the collection unit can prioritize collecting only information that can be acquired in a short time. By adjusting the collection timing based on the user's emotions, the collection unit can collect information at more appropriate times. Emotion estimation is realized using emotion engines or generative AI, such as text generation AI (e.g., LLM) or multimodal generative AI, but is not limited thereto. Specifically, the collection unit receives diverse input data for emotion estimation, such as audio data (e.g., one-minute audio waveform, 16 kHz sampling, monaural PCM), facial image data (e.g., 640×480 pixel RGB image), and text data (e.g., user diary or SNS post string data, up to 512 tokens). The collection unit applies an audio emotion recognition model (e.g., hybrid of convolutional neural network and recurrent neural network), facial expression recognition model (e.g., ResNet-based CNN), and text emotion classification model (e.g., Transformer-based large language model) to each modality, outputting emotion labels (e.g., stress, relaxation, excitement, hurry) and emotion scores (e.g., stress level 0.85, relaxation level 0.12, real values from 0 to 1). The collection unit integrates these emotion estimation results, combines them with time, situation, and past emotion history, and executes a collection timing optimization algorithm (e.g., lowering collection frequency when stress level is high, collecting within 30 minutes after exercise when relaxation level is high, rule-based branching). For example, input examples include “audio: calm tone, facial image: smile, text: I was able to relax today” or “audio: fast and high-pitched, facial image: frown, text: busy at work,” and output examples include “emotion: relaxation, collection timing: within 30 minutes after exercise” or “emotion: stress, collection timing: nighttime relaxation time.” The collection unit automatically adjusts the timing of collecting health and exercise information (e.g., heart rate, step count, calories burned, sleep duration) in conjunction with the estimated emotions, minimizing user burden and stress while achieving optimal data acquisition. For emotion estimation model training, annotated emotion datasets for each modality are used, applying cross-entropy loss or multitask loss as the loss function, and optimizing weights by gradient descent. Data augmentation such as pitch shifting for audio, horizontal flipping for images, and synonym replacement for text is performed to improve generalization performance. Unlike conventional subjective timing adjustment or simple scheduled collection by humans, the collection unit implements computer-specific non-conventional procedures such as multimodal emotion estimation, rule-based branching, and automatic timing optimization in high-dimensional feature space, thereby achieving significant improvements in collection accuracy, user experience, and data quality. Specific application fields include stress management support applications, mental health monitoring systems, and personalized health management services.

The collection unit can analyze a user's past health checkup results and select an optimal collection method. For example, the collection unit determines the types of data to be collected based on specific health indicators from the user's past health checkup results. The collection unit can also set a regular collection schedule based on the user's past health checkup results. Furthermore, the collection unit can perform data collection focused on specific health indicators based on the user's health checkup results. By analyzing past health checkup results, the collection unit can select an optimal collection method. Specifically, the collection unit acquires more than one year of past health checkup data for each user (e.g., time-series vector data for blood pressure, blood glucose level, cholesterol level, body weight, BMI, liver function values, up to 12 samples per indicator) from a database. The collection unit performs statistical preprocessing such as moving average, trend scoring, and anomaly detection (e.g., outlier detection by Z-score) on these time-series data, automatically extracting indicators with high health risk (e.g., blood pressure exceeding reference value, rising trend in blood glucose level). For each extracted risk indicator, the collection unit determines the types of data to be collected (e.g., for blood pressure risk: blood pressure, heart rate, salt intake; for blood glucose risk: blood glucose level, meal content, exercise amount), and applies an algorithm to optimize collection frequency (e.g., daily for high-risk indicators, weekly for low-risk indicators) and collection timing (e.g., before breakfast, after exercise). For example, input examples include “average blood pressure over the past 12 months: 135/90 mmHg, blood glucose level: stable, cholesterol level: rising trend,” and output examples include “priority collection indicators: blood pressure, cholesterol; collection frequency: blood pressure every morning, cholesterol once a month.” The collection unit automatically generates a collection schedule for each user and sends collection instructions to wearable devices or linked apps. Furthermore, the collection unit dynamically reconfigures the collection method according to changes in health checkup results, adaptively controlling collection frequency, such as reducing frequency when risk decreases. For health checkup data analysis, machine learning models such as random forest or gradient boosting decision trees are used to calculate risk prediction scores, and branching control is implemented in a threshold judgment unit to monitor only high-risk indicators. Unlike conventional uniform regular collection or indicator selection based on human experience, the collection unit implements computer-specific non-conventional procedures such as high-dimensional time-series analysis, automatic risk extraction, and adaptive collection scheduling, thereby achieving significant improvements in collection efficiency, risk detection accuracy, and data management optimization. Specific application fields include chronic disease management systems, corporate health management support services, and patient monitoring platforms for medical institutions.

The collection unit can perform filtering based on a user's current living conditions and areas of interest when collecting health and exercise information. For example, if the user is on a diet, the collection unit prioritizes collecting information related to calorie consumption. If the user is undergoing medical treatment, the collection unit focuses on collecting information related to health conditions. Furthermore, if the user is a professional athlete, the collection unit prioritizes collecting information related to exercise performance. By filtering information based on the user's living conditions and areas of interest, the collection unit can collect more relevant information. Specifically, the collection unit receives user profile data (e.g., status flags such as on a diet, undergoing medical treatment, athlete, target values, interest tags as structured data) as input. The collection unit implements a filtering algorithm that calculates priority scores for collection target data (e.g., heart rate, step count, calories burned, sleep duration, meal content, exercise intensity) in conjunction with profile information (e.g., prioritizing calories burned and step count during dieting, prioritizing blood pressure, blood glucose level, and body temperature during medical treatment, prioritizing exercise intensity and muscle fatigue for athletes). Based on the priority scores, the collection unit automatically selects collection frequency, timing, and devices (e.g., wearable devices, smartphone apps, linked medical devices), suppressing unnecessary data collection. For example, input examples include “user status: on a diet, target: burn 2,000 kcal per day, area of interest: aerobic exercise” or “user status: undergoing medical treatment, target: normalize blood pressure, area of interest: low-salt diet,” and output examples include “collection targets: calories burned, step count, meal content; collection frequency: after each meal and exercise” or “collection targets: blood pressure, salt intake, body temperature; collection frequency: morning and evening.” The collection unit detects changes in the user's living conditions and areas of interest in real time and dynamically updates filtering rules to maintain optimal information collection at all times. Furthermore, the collection unit can accumulate user feedback and achievement levels as learning data and optimize filtering algorithm parameters using reinforcement learning. Unlike conventional uniform information collection or manual selection by humans, the collection unit implements computer-specific non-conventional procedures such as high-dimensional profile analysis, automatic priority assignment, and adaptive filtering, thereby achieving significant improvements in collection efficiency, relevance, and user satisfaction. Specific application fields include personalized health management applications, diet support services, and training monitoring systems for athletes.

The collection unit can estimate a user's emotions and determine the priority of health and exercise information to be collected based on the estimated emotions. For example, if the user is feeling stressed, the collection unit prioritizes collecting information related to relaxation. If the user is relaxed, the collection unit can also prioritize collecting information related to exercise performance. Furthermore, if the user is in a hurry, the collection unit can prioritize collecting information that can be acquired in a short time. By determining the priority of information based on the user's emotions, the collection unit can prioritize collecting more important information. Emotion estimation is realized using emotion engines or generative AI, such as text generation AI (e.g., LLM) or multimodal generative AI, but is not limited thereto. Specifically, the collection unit receives diverse input data for emotion estimation, such as audio data (e.g., one-minute audio waveform, 16 kHz sampling), facial image data (e.g., 640×480 pixel RGB image), and text data (e.g., SNS posts or diary strings, up to 512 tokens). The collection unit applies an audio emotion recognition model (e.g., CNN+RNN), facial expression recognition model (e.g., ResNet-based CNN), and text emotion classification model (e.g., Transformer-based large language model), outputting emotion labels (e.g., stress, relaxation, hurry) and emotion scores (e.g., stress level 0.8, relaxation level 0.1). According to the estimated emotions, the collection unit implements a priority determination algorithm that determines the priority of collection target data (e.g., relaxation-related information during stress, exercise performance information during relaxation, information that can be collected quickly when in a hurry) based on emotion scores and threshold judgment branching. For example, input examples include “audio: tense tone, facial image: frown, text: work is tough” or “audio: calm tone, facial image: smile, text: exercised today,” and output examples include “emotion: stress, priority collection: relaxation-related information” or “emotion: relaxation, priority collection: exercise performance information.” The collection unit automatically adjusts collection frequency, timing, and devices according to priority, realizing information collection optimized for the user's state. For emotion estimation model training, annotated emotion datasets for each modality are used, applying cross-entropy loss or multitask loss, and optimizing weights by gradient descent. Unlike conventional subjective priority assignment or uniform information collection by humans, the collection unit implements computer-specific non-conventional procedures such as multimodal emotion estimation, automatic priority determination, and adaptive information collection, thereby achieving significant improvements in collection efficiency, relevance, and user experience. Specific application fields include stress management support applications, personalized health management services, and mental health monitoring systems.

The collection unit can prioritize the collection of highly relevant information by considering a user's geographic location when collecting health and exercise information. For example, if the user is in a specific region, the collection unit prioritizes collecting information related to the climate or environment of that region. If the user is traveling, the collection unit can prioritize collecting information related to health risks at the travel destination. Furthermore, if the user is in a specific exercise facility, the collection unit can prioritize collecting information related to exercise performance at that facility. By considering the user's geographic location, the collection unit can prioritize collecting highly relevant information. Specifically, the collection unit acquires the user's current location information (e.g., GPS coordinates, latitude and longitude data, location accuracy within 5 meters) in real time and links with a geographic information database (e.g., climate data by region, environmental risk information, facility information). The collection unit implements a geographic information filtering algorithm that calculates priority scores for collection target data (e.g., oxygen saturation and heart rate at high altitudes, air pollution index and allergen information in urban areas, exercise intensity and calories burned at exercise facilities) in conjunction with location information (e.g., weighting based on region-specific risks, branching by facility type). Based on the priority scores, the collection unit automatically selects collection frequency, timing, and devices, suppressing unnecessary data collection. For example, input examples include “current location: high altitude at 2,000 meters, temperature 5° C., facility: mountain trail” or “current location: urban area, high PM2.5, facility: gym,” and output examples include “priority collection: oxygen saturation, heart rate; collection frequency: every 30 minutes” or “priority collection: air pollution index, exercise intensity; collection frequency: before and after exercise.” The collection unit detects user movement and location changes in real time and dynamically updates geographic information filtering rules to maintain optimal information collection at all times. Furthermore, the collection unit learns the relationship between geographic information and health risks using machine learning models (e.g., random forest, gradient boosting decision trees), calculates risk prediction scores, and implements branching control in a threshold judgment unit. Unlike conventional manual location input or uniform information collection by humans, the collection unit implements computer-specific non-conventional procedures such as real-time location information analysis, automatic priority assignment, and adaptive information collection, thereby achieving significant improvements in collection efficiency, relevance, and risk detection accuracy. Specific application fields include health management applications for travelers, environmental risk monitoring systems, and health support services linked to exercise facilities.

The collection unit can analyze a user's social media activity and collect relevant information when collecting health and exercise information. For example, the collection unit collects information related to exercise performance based on exercise records shared by the user on social media. The collection unit can also collect information related to dietary trends based on meal photographs shared by the user on social media. Furthermore, the collection unit can collect information related to health conditions based on health-related posts shared by the user on social media. By analyzing the user's social media activity, the collection unit can collect relevant information. Specifically, with the user's permission, the collection unit automatically acquires post data (e.g., text posts, images, videos, shared exercise records) from major social media platforms via API. The collection unit receives text data (e.g., post text up to 512 tokens), image data (e.g., 640×480 pixel JPEG images), and exercise record data (e.g., date, activity type, distance, calories burned as structured data) as input, and applies a text analysis model (e.g., Transformer-based large language model), image analysis model (e.g., CNN for meal image classification), and exercise record analysis model (e.g., time-series clustering algorithm). The collection unit automatically extracts exercise performance indicators (e.g., running distance, calories burned), dietary trends (e.g., high-fat, low-carb, vegetable-centered), and health conditions (e.g., poor physical condition, fatigue, good condition) from post content, and calculates priority scores for collection target data. For example, input examples include “post: ran 10 km today, image: photo of salad and steak” or “post: feeling tired lately, image: photo of fast food,” and output examples include “priority collection: exercise performance, meal content; collection frequency: after exercise and meals” or “priority collection: health condition, dietary trends; collection frequency: daily.” The collection unit detects changes in social media activity in real time and dynamically updates information collection rules to always collect the latest and most relevant information. Furthermore, the collection unit accumulates user posting trends and feedback as learning data and continuously optimizes analysis model parameters. Unlike conventional manual post analysis or uniform information collection by humans, the collection unit implements computer-specific non-conventional procedures such as multimodal post analysis, automatic priority assignment, and adaptive information collection, thereby achieving significant improvements in collection efficiency, relevance, and user experience. Specific application fields include SNS-linked health management applications, dietary and exercise trend analysis services, and personalized health support platforms.

The analysis unit can estimate a user's emotions and adjust the manner of presenting analysis results based on the estimated emotions. For example, if the user is nervous, the analysis unit provides simple and highly visible analysis results. If the user is relaxed, the analysis unit can also provide detailed analysis results. Furthermore, if the user is in a hurry, the analysis unit can provide concise analysis results that focus on key points. By adjusting the manner of presenting analysis results based on the user's emotions, the analysis unit can provide more appropriate analysis results. Emotion estimation is realized using emotion engines or generative AI, such as text generation AI (e.g., LLM) or multimodal generative AI, but is not limited thereto. Specifically, the analysis unit receives diverse input data for emotion estimation, such as audio data (e.g., one-minute audio waveform, 16 kHz sampling, monaural PCM), facial image data (e.g., 640×480 pixel RGB image), and text data (e.g., SNS posts or diary strings, up to 512 tokens). The analysis unit applies an audio emotion recognition model (e.g., hybrid of convolutional neural network and recurrent neural network), facial expression recognition model (e.g., ResNet-based CNN), and text emotion classification model (e.g., Transformer-based large language model) to each modality, outputting emotion labels (e.g., nervousness, relaxation, hurry) and emotion scores (e.g., nervousness level 0.75, relaxation level 0.15, real values from 0 to 1). The analysis unit integrates these emotion estimation results, combines them with time, situation, and past emotion history, and executes an analysis expression optimization algorithm (e.g., reducing the number of elements and suppressing color and graph expression when nervousness level is high, adding detailed graphs and annotations when relaxation level is high, presenting only key points in bullet points when in a hurry, rule-based branching). For example, input examples include “audio: fast and high-pitched, facial image: frown, text: busy at work” or “audio: calm tone, facial image: smile, text: I was able to relax today,” and output examples include “emotion: nervousness, analysis expression: simple numerical list” or “emotion: relaxation, analysis expression: detailed graph and annotated report.” The analysis unit automatically adjusts the output format (e.g., text summary, graph, heatmap, interactive UI) and level of detail (e.g., number of items, amount of annotation, color intensity) of analysis results according to the estimated emotions, optimizing user experience. For emotion estimation model training, annotated emotion datasets for each modality are used, applying cross-entropy loss or multitask loss as the loss function, and optimizing weights by gradient descent. Data augmentation such as pitch shifting for audio, horizontal flipping for images, and synonym replacement for text is performed to improve generalization performance. Unlike conventional subjective expression adjustment or uniform analysis output by humans, the analysis unit implements computer-specific non-conventional procedures such as multimodal emotion estimation, rule-based branching, and automatic expression optimization in high-dimensional feature space, thereby achieving significant improvements in analysis accuracy, user experience, and information transmission efficiency. Specific application fields include personalized health management dashboards, stress management support systems, and patient explanation support tools for medical institutions.

The analysis unit can adjust the level of detail of analysis based on the importance of health and exercise information during analysis. For example, the analysis unit performs detailed analysis for important health indicators. The analysis unit can also perform detailed analysis of exercise performance information according to the user's goals. Furthermore, the analysis unit can perform detailed analysis of dietary trend information according to health conditions. By adjusting the level of detail of analysis based on the importance of health and exercise information, the analysis unit can perform more detailed analysis of important information. Specifically, the analysis unit receives as input health, exercise, and dietary data (e.g., blood pressure, blood glucose level, step count, calories burned, image feature vectors of meal photographs) from the collection unit. The analysis unit implements an importance evaluation algorithm that calculates importance scores for each data item (e.g., blood pressure 0.9, step count 0.6, dietary trend 0.8), weighting factors such as user health risk, physician instructions, and past anomaly detection history. For items with high importance scores, the analysis unit performs detailed analysis (e.g., time-series trend analysis, anomaly detection, factor decomposition, graphing, annotation), and for items with low importance scores, the analysis unit performs summary analysis (e.g., presenting only mean and median values, simple graphs). For example, input examples include “blood pressure: 140/90 mmHg (importance 0.95), step count: 5,000 steps (importance 0.5), dietary trend: high fat (importance 0.8),” and output examples include “blood pressure: trend graph for the past year, anomaly warning, lifestyle advice; step count: weekly average only; dietary trend: detailed nutrient breakdown and recommended improvements.” The analysis unit dynamically adjusts the weighting of importance scores according to the user's goals (e.g., diet, medical treatment, muscle strengthening) and health conditions (e.g., caution, needs improvement), optimizing the level of detail of analysis. Importance evaluation models use machine learning models such as random forest or gradient boosting decision trees, optimizing parameters using past analysis results and user feedback as learning data. Unlike conventional uniform analysis or detail level setting based on human experience, the analysis unit implements computer-specific non-conventional procedures such as automatic importance evaluation and detail level optimization in high-dimensional feature space, thereby achieving significant improvements in analysis efficiency, accuracy, and user satisfaction. Specific application fields include chronic disease management systems, personalized health report generation services, and patient monitoring tools for medical institutions.

The analysis unit can apply different analysis algorithms according to categories of health and exercise information during analysis. For example, the analysis unit applies health indicator analysis algorithms to information related to health conditions. The analysis unit can also apply exercise analysis algorithms to information related to exercise performance. Furthermore, the analysis unit can apply dietary analysis algorithms to information related to dietary trends. By applying different analysis algorithms according to categories of health and exercise information, the analysis unit can provide more appropriate analysis results. Specifically, the analysis unit receives as input data for each category from the collection unit (e.g., health indicators as numerical vectors for blood pressure, blood glucose level, body weight; exercise data as time-series arrays for step count, calories burned, exercise intensity; dietary data as image feature vectors and estimated calorie values). The analysis unit applies health condition classification algorithms (e.g., random forest for classification into healthy, caution, needs improvement) to health indicator data, time-series analysis algorithms (e.g., recurrent neural network for extracting patterns of exercise frequency and intensity) to exercise data, and image analysis plus nutrient estimation algorithms (e.g., convolutional neural network for meal image encoding plus nutrient regression model) to dietary data. For example, input examples include “health indicators: blood pressure 130/85 mmHg, blood glucose 110 mg/dL; exercise data: one week of step count and calories burned; meal image: pasta and salad,” and output examples include “health condition: caution; exercise status: standard; dietary trend: high carbohydrate.” The analysis unit automatically selects different algorithms for each category and integrates analysis results to generate a comprehensive health report. Algorithm selection logic uses category determination rules or meta-learning models, applying optimal analysis methods according to data type and quality. Unlike conventional uniform analysis or simple aggregation by humans, the analysis unit implements computer-specific non-conventional procedures such as category determination for high-dimensional data, automatic algorithm selection, and integration of diverse analysis methods, thereby achieving significant improvements in analysis accuracy, flexibility, and scalability. Specific application fields include integrated health management platforms, performance analysis systems for athletes, and dietary trend analysis services.

The analysis unit can estimate a user's emotions and adjust the length of analysis based on the estimated emotions. For example, if the user is in a hurry, the analysis unit provides concise analysis results that focus on key points. If the user is relaxed, the analysis unit can also provide detailed analysis results. Furthermore, if the user is excited, the analysis unit can provide visually stimulating analysis results. By adjusting the length of analysis based on the user's emotions, the analysis unit can provide more appropriate analysis results. Emotion estimation is realized using emotion engines or generative AI, such as text generation AI (e.g., LLM) or multimodal generative AI, but is not limited thereto. Specifically, the analysis unit receives diverse input data for emotion estimation, such as audio data (e.g., one-minute audio waveform, 16 kHz sampling), facial image data (e.g., 640×480 pixel RGB image), and text data (e.g., SNS posts or diary strings, up to 512 tokens). The analysis unit applies an audio emotion recognition model (e.g., CNN+RNN), facial expression recognition model (e.g., ResNet-based CNN), and text emotion classification model (e.g., Transformer-based large language model), outputting emotion labels (e.g., hurry, relaxation, excitement) and emotion scores (e.g., hurry level 0.8, relaxation level 0.1). According to the estimated emotions, the analysis unit implements a length optimization algorithm that automatically adjusts the length of analysis results (e.g., short sentences with key points only, detailed graphs and annotated reports, visually emphasized output with colors and animations) by selecting output templates based on emotion scores and threshold judgment branching. For example, input examples include “audio: fast speech, facial image: wide-open eyes, text: in a hurry” or “audio: calm tone, facial image: smile, text: I was able to relax today,” and output examples include “emotion: hurry, analysis length: key points only” or “emotion: relaxation, analysis length: detailed report.” By optimizing the length and format of analysis results for the user's state, the analysis unit significantly improves information transmission efficiency, user satisfaction, and utilization rate of analysis. For emotion estimation model training, annotated emotion datasets for each modality are used, applying cross-entropy loss or multitask loss, and optimizing weights by gradient descent. Unlike conventional subjective length adjustment or uniform analysis output by humans, the analysis unit implements computer-specific non-conventional procedures such as multimodal emotion estimation, automatic length optimization, and adaptive analysis output, thereby achieving significant improvements in analysis efficiency, user experience, and information transmission accuracy. Specific application fields include personalized health report generation services, stress management support tools, and patient explanation support systems for medical institutions.

The analysis unit can determine the priority of analysis based on the timing of health and exercise information collection during analysis. For example, the analysis unit prioritizes the analysis of the latest health checkup results. Additionally, the analysis unit can prioritize the analysis of recent exercise records. Furthermore, the analysis unit can also prioritize the analysis of the latest meal photographs. By determining the priority of analysis based on the timing of health and exercise information collection, the analysis unit can preferentially analyze the most recent information. Specifically, the analysis unit receives health, exercise, and meal data (e.g., health checkup results as date-stamped numerical vectors, exercise records as time-series arrays, meal photographs as timestamped image data) from the collection unit as input. The analysis unit extracts the collection timing of each data item (e.g., timestamp, date, time) and applies a latest-data-priority algorithm (e.g., prioritizing data from the past week, summarizing older data only, etc.). The analysis unit performs detailed analysis (e.g., anomaly detection, trend analysis, nutrient estimation, etc.) in order from the prioritized data, and treats older data as summarized analysis or reference information. For example, input examples include “Health checkup result: 2024 Jun. 1, Exercise record: 2024 Jun. 3, Meal photograph: 2024 Jun. 2,” and output examples include “Priority analysis: Exercise record→Meal photograph→Health checkup result.” The analysis unit can also dynamically change the rules for determining analysis priority (e.g., prioritizing health checkup results, analyzing exercise records daily, etc.) according to user settings or instructions from medical institutions. The priority determination model may use rule-based branching or machine learning models (e.g., decision trees, random forests, etc.), optimizing parameters using past analysis results and user feedback as training data. Unlike conventional human-based uniform analysis order or experience-based priority settings, the analysis unit implements computer-specific, non-conventional procedures such as high-dimensional time-series data analysis, automatic priority assignment, and adaptive analysis order control, thereby achieving significant technical effects such as improved analysis efficiency, information freshness, and user satisfaction. Specific application fields include real-time health monitoring systems, personalized health report generation services, and patient management platforms for medical institutions.

The analysis unit can adjust the order of analysis based on the relevance of health and exercise information during analysis. For example, the analysis unit prioritizes the analysis of information related to health conditions. Additionally, the analysis unit can prioritize the analysis of information related to exercise performance. Furthermore, the analysis unit can also prioritize the analysis of information related to dietary trends. By adjusting the order of analysis based on the relevance of health and exercise information, the analysis unit can preferentially analyze more relevant information. Specifically, the analysis unit receives health, exercise, and meal data (e.g., health indicator vectors, exercise time-series arrays, meal image feature vectors, etc.) from the collection unit as input. The analysis unit implements a relevance evaluation algorithm (e.g., Pearson correlation, principal component analysis, graph-based clustering, etc.) that calculates relevance scores between data items (e.g., correlation coefficient between health conditions and exercise performance, degree of correlation between dietary trends and health indicators, etc.). The analysis unit prioritizes the analysis of items with high relevance scores, while items with low relevance are analyzed later or only summarized. For example, input examples include “Health condition: caution required, Exercise status: low activity, Dietary trend: high fat,” and output examples include “Priority analysis order: Health condition→Dietary trend→Exercise status.” The analysis unit can dynamically adjust the weighting of relevance evaluation and analysis order rules according to user goals or instructions from medical institutions. The relevance evaluation model may use machine learning models (e.g., random forests, graph neural networks, etc.), optimizing parameters using past analysis results and user feedback as training data. Unlike conventional human-based experience or uniform analysis order settings, the analysis unit implements computer-specific, non-conventional procedures such as high-dimensional data relevance analysis, automatic order optimization, and adaptive analysis control, thereby achieving significant technical effects such as improved analysis efficiency, information value, and user satisfaction. Specific application fields include integrated health management systems, personalized health report generation services, and patient monitoring tools for medical institutions.

The proposal unit can estimate a user's emotions and adjust the manner of presenting proposals based on the estimated emotions. For example, if the user is nervous, the proposal unit provides simple and highly visible proposals. Additionally, if the user is relaxed, the proposal unit can provide detailed proposals. Furthermore, if the user is in a hurry, the proposal unit can provide concise proposals focusing on key points. By adjusting the manner of presenting proposals based on the user's emotions, the proposal unit can provide more appropriate proposals. Emotion estimation is realized using emotion engines or generative AI, such as text generation AI (e.g., LLMs) or multimodal generative AI, but is not limited to these examples. Specifically, the proposal unit receives various input data for emotion estimation, such as voice data (e.g., 1-minute audio waveform, 16 kHz sampling, monaural PCM), facial image data (e.g., 640×480 pixel RGB images), and text data (e.g., SNS posts or diary entries, up to 512 tokens). The proposal unit applies a voice emotion recognition model (e.g., hybrid configuration of convolutional neural networks and recurrent neural networks), a facial expression recognition model (e.g., ResNet-based CNN), and a text emotion classification model (e.g., Transformer-based large language model), outputting emotion labels (e.g., nervous, relaxed, hurried, etc.) and emotion scores (e.g., nervousness 0.75, relaxation 0.15, etc., as real values from 0 to 1) for each modality. The proposal unit integrates these emotion estimation results, combines them with time, context, and past emotion history, and executes a proposal expression optimization algorithm (e.g., reducing the number of elements and suppressing color and graph expressions when nervousness is high, adding detailed graphs and annotations when relaxation is high, presenting only key points in bullet form when hurried, etc., using rule-based branching). For example, input examples include “Voice: fast and high-pitched, Facial image: frown, Text: busy at work” or “Voice: calm tone, Facial image: smile, Text: had a relaxing day,” and output examples include “Emotion: nervous, Proposal expression: simple numerical list” and “Emotion: relaxed, Proposal expression: detailed report with graphs and annotations.” The proposal unit automatically adjusts the output format (e.g., text summary, graph, heatmap, interactive UI, etc.) and level of detail (e.g., number of items, amount of annotation, color intensity) of proposal results according to the estimated emotions, optimizing the user experience. For training the emotion estimation model, annotated emotion datasets are used for each modality, applying cross-entropy loss or multitask loss as the loss function, and optimizing weights using gradient descent. Furthermore, data augmentation such as pitch shifting for audio, horizontal flipping for images, and synonym replacement for text is performed to improve generalization performance. Unlike conventional human-based subjective expression adjustment or uniform proposal output, the proposal unit implements computer-specific, non-conventional procedures such as multimodal emotion estimation, rule-based branching, and automatic expression optimization in high-dimensional feature space, thereby achieving significant technical effects such as improved proposal accuracy, user experience, and information transmission efficiency. Specific application fields include personalized health management dashboards, stress management support systems, and patient explanation support tools for medical institutions.

The proposal unit can adjust the level of detail of proposals based on the importance of cooking recipes during proposal. For example, the proposal unit provides detailed proposals for cooking recipes containing important nutrients. Additionally, for people on a diet, the proposal unit can provide detailed proposals for calorie information. Furthermore, for people undergoing medical treatment, the proposal unit can provide proposals emphasizing nutritional balance. By adjusting the level of detail of proposals based on the importance of cooking recipes, the proposal unit can provide more detailed proposals for more important information. Specifically, the proposal unit receives feature vectors such as health conditions, exercise status, and dietary trends from the analysis unit, as well as importance scores assigned to each cooking recipe in the recipe database (e.g., nutrient content, physician recommendation level, degree of match with user goals, etc.). The proposal unit implements an importance evaluation algorithm (e.g., weighting by nutrient, scoring according to user status, matching with past health risk history), and generates detailed proposals (e.g., nutrient breakdown tables, detailed cooking procedures, ingredient origin information, allergen information, recommended intake timing, etc.) for recipes with high importance scores. For example, input examples include “User status: on a diet, Goal: 1800 kcal/day, Important nutrients: protein, dietary fiber” or “User status: undergoing medical treatment, Goal: optimal PFC balance, Important nutrients: vitamins, minerals,” and output examples include “Recommended recipe: steamed chicken breast and vegetables (with detailed nutrient breakdown), tofu salad (with calorie, PFC balance, and allergen information).” For recipes with low importance, the proposal unit generates summarized proposals (e.g., only recipe name and calories, simple ingredient list, etc.). The importance evaluation model may use machine learning models such as random forests or gradient boosting decision trees, optimizing parameters using past proposal history and user feedback as training data. Unlike conventional human-based uniform proposals or experience-based detail settings, the proposal unit implements computer-specific, non-conventional procedures such as automatic importance evaluation and detail optimization in high-dimensional feature space, thereby achieving significant technical effects such as improved proposal efficiency, accuracy, and user satisfaction. Specific application fields include chronic disease management support applications, personalized nutrition guidance services, and meal proposal systems for medical institutions.

The proposal unit can apply different proposal algorithms according to the category of cooking recipes during proposal. For example, the proposal unit applies a calorie restriction algorithm to diet recipes. Additionally, the proposal unit can apply a nutritional balance algorithm to recipes for people undergoing medical treatment. Furthermore, the proposal unit can apply a high-protein algorithm to recipes for professional athletes. By applying different proposal algorithms according to the category of cooking recipes, the proposal unit can provide more appropriate proposals. Specifically, the proposal unit receives feature vectors such as the user's health condition, exercise status, and dietary trends from the analysis unit, as well as category information assigned to each recipe in the recipe database (e.g., diet, medical treatment, athlete, general meal, etc.). The proposal unit implements category determination logic (e.g., rule-based branching or meta-learning models based on user goals, health condition, exercise intensity, physician instructions, etc.), and automatically selects and applies the optimal proposal algorithm for each category (e.g., for the diet category, a calorie restriction algorithm prioritizing meals under 500 kcal and low fat; for the medical treatment category, a nutritional balance algorithm emphasizing PFC balance, vitamins, and minerals; for the athlete category, a high-protein algorithm emphasizing at least 30 g of protein per meal and muscle recovery components, etc.). For example, input examples include “User status: on a diet, Goal: 1800 kcal/day,” “User status: undergoing medical treatment, Goal: optimal PFC balance,” “User status: athlete, Goal: muscle strength enhancement,” and output examples include “Recommended recipe: low-calorie Japanese meal set (calorie restriction algorithm applied), grilled salmon in foil (nutritional balance algorithm applied), grilled chicken and bean salad (high-protein algorithm applied).” The proposal unit automatically adjusts parameters and output formats (e.g., nutrient breakdown tables, detailed cooking procedures, recommended intake timing, etc.) for each category to generate optimal proposals for the user. The algorithm selection logic may use category determination rules or meta-learning models, applying the optimal proposal method according to the type and quality of data. Unlike conventional human-based uniform proposals or simple recipe selection, the proposal unit implements computer-specific, non-conventional procedures such as high-dimensional data category determination, automatic algorithm selection, and integration of diverse proposal methods, thereby achieving significant technical effects such as improved proposal accuracy, flexibility, and scalability. Specific application fields include integrated health management platforms, nutrition proposal systems for athletes, and medical meal support services.

The proposal unit can estimate a user's emotions and adjust the length of proposals based on the estimated emotions. For example, if the user is in a hurry, the proposal unit provides short and concise proposals focusing on key points. Additionally, if the user is relaxed, the proposal unit can provide detailed proposals. Furthermore, if the user is excited, the proposal unit can provide visually stimulating proposals. By adjusting the length of proposals based on the user's emotions, the proposal unit can provide more appropriate proposals. Emotion estimation is realized using emotion engines or generative AI, such as text generation AI (e.g., LLMs) or multimodal generative AI, but is not limited to these examples. Specifically, the proposal unit receives various input data for emotion estimation, such as voice data (e.g., 1-minute audio waveform, 16 kHz sampling), facial image data (e.g., 640×480 pixel RGB images), and text data (e.g., SNS posts or diary entries, up to 512 tokens). The proposal unit applies a voice emotion recognition model (e.g., CNN+RNN), a facial expression recognition model (e.g., ResNet-based CNN), and a text emotion classification model (e.g., Transformer-based large language model), outputting emotion labels (e.g., hurried, relaxed, excited, etc.) and emotion scores (e.g., hurry level 0.8, relaxation level 0.1, etc.). The proposal unit implements a length optimization algorithm that automatically adjusts the length of proposal results (e.g., short sentences with only key points, detailed reports with graphs and annotations, visually emphasized output with colors and animations, etc.) according to the estimated emotions (e.g., output template selection based on emotion scores, branching by threshold judgment). For example, input examples include “Voice: fast, Facial image: wide-open eyes, Text: in a hurry” or “Voice: calm tone, Facial image: smile, Text: had a relaxing day,” and output examples include “Emotion: hurried, Proposal length: key points only” and “Emotion: relaxed, Proposal length: detailed report.” By optimizing the length and format of proposal results according to the user's state, the proposal unit greatly improves information transmission efficiency, user satisfaction, and proposal utilization rate. For training the emotion estimation model, annotated emotion datasets are used for each modality, applying cross-entropy loss or multitask loss, and optimizing weights using gradient descent. Unlike conventional human-based subjective length adjustment or uniform proposal output, the proposal unit implements computer-specific, non-conventional procedures such as multimodal emotion estimation, automatic length optimization, and adaptive proposal output, thereby achieving significant technical effects such as improved proposal efficiency, user experience, and information transmission accuracy. Specific application fields include personalized health report generation services, stress management support tools, and patient explanation support systems for medical institutions.

The proposal unit can determine the priority of proposals based on the submission timing of cooking recipes during proposal. For example, the proposal unit provides proposals based on the latest health checkup results. Additionally, the proposal unit can provide proposals based on recent exercise records. Furthermore, the proposal unit can provide proposals based on the latest meal photographs. By determining the priority of proposals based on the submission timing of cooking recipes, the proposal unit can preferentially provide proposals based on the most recent information. Specifically, the proposal unit receives health, exercise, and meal data (e.g., health checkup results as date-stamped numerical vectors, exercise records as time-series arrays, meal photographs as timestamped image data) from the analysis unit as input. The proposal unit extracts the collection timing of each data item (e.g., timestamp, date, time) and applies a latest-data-priority algorithm (e.g., prioritizing data from the past week, summarizing older data only, etc.). The proposal unit generates detailed recipe proposals in order from the prioritized data (e.g., recipes optimizing nutritional balance based on the latest health checkup results, recipes supplementing calories consumed based on recent exercise records, recipes improving dietary trends based on the latest meal photographs, etc.), and treats older data as summarized proposals or reference information. For example, input examples include “Health checkup result: 2024 Jun. 1, Exercise record: 2024 Jun. 3, Meal photograph: 2024 Jun. 2,” and output examples include “Priority proposal: Exercise record→Meal photograph→Health checkup result.” The proposal unit can also dynamically change the rules for determining proposal priority (e.g., prioritizing health checkup results, proposing exercise records daily, etc.) according to user settings or instructions from medical institutions. The priority determination model may use rule-based branching or machine learning models (e.g., decision trees, random forests, etc.), optimizing parameters using past proposal results and user feedback as training data. Unlike conventional human-based uniform proposal order or experience-based priority settings, the proposal unit implements computer-specific, non-conventional procedures such as high-dimensional time-series data analysis, automatic priority assignment, and adaptive proposal order control, thereby achieving significant technical effects such as improved proposal efficiency, information freshness, and user satisfaction. Specific application fields include real-time health monitoring systems, personalized health report generation services, and patient management platforms for medical institutions.

The proposal unit can adjust the order of proposals based on the relevance of cooking recipes during proposal. For example, the proposal unit prioritizes proposals related to health conditions. Additionally, the proposal unit can prioritize proposals related to exercise performance. Furthermore, the proposal unit can prioritize proposals related to dietary trends. By adjusting the order of proposals based on the relevance of cooking recipes, the proposal unit can preferentially provide more relevant information. Specifically, the proposal unit receives health, exercise, and meal data (e.g., health indicator vectors, exercise time-series arrays, meal image feature vectors, etc.) from the analysis unit as input. The proposal unit implements a relevance evaluation algorithm (e.g., Pearson correlation, principal component analysis, graph-based clustering, etc.) that calculates relevance scores between data items (e.g., correlation coefficient between health conditions and exercise performance, degree of correlation between dietary trends and health indicators, etc.). The proposal unit prioritizes proposals for items with high relevance scores, while items with low relevance are proposed later or only summarized. For example, input examples include “Health condition: caution required, Exercise status: low activity, Dietary trend: high fat,” and output examples include “Priority proposal order: Health condition→Dietary trend→Exercise status.” The proposal unit can dynamically adjust the weighting of relevance evaluation and proposal order rules according to user goals or instructions from medical institutions. The relevance evaluation model may use machine learning models (e.g., random forests, graph neural networks, etc.), optimizing parameters using past proposal results and user feedback as training data. Unlike conventional human-based experience or uniform proposal order settings, the proposal unit implements computer-specific, non-conventional procedures such as high-dimensional data relevance analysis, automatic order optimization, and adaptive proposal control, thereby achieving significant technical effects such as improved proposal efficiency, information value, and user satisfaction. Specific application fields include integrated health management systems, personalized health report generation services, and patient monitoring tools for medical institutions.

The ordering unit can estimate a user's emotions and adjust the method of ordering ingredients based on the estimated emotions. For example, if the user feels stressed, the ordering unit provides a simple ordering interface. Additionally, if the user is relaxed, the ordering unit can provide detailed ordering options. Furthermore, if the user is in a hurry, the ordering unit can provide a method for quick ordering. By adjusting the method of ordering ingredients based on the user's emotions, the ordering unit can provide more appropriate ordering methods. Emotion estimation is realized using emotion engines or generative AI, such as text generation AI (e.g., LLMs) or multimodal generative AI, but is not limited to these examples. Specifically, the ordering unit receives various input data for emotion estimation, such as voice data (e.g., 1-minute audio waveform, 16 kHz sampling, monaural PCM), facial image data (e.g., 640×480 pixel RGB images), and text data (e.g., SNS posts or diary entries, up to 512 tokens). The ordering unit applies a voice emotion recognition model (e.g., hybrid configuration of convolutional neural networks and recurrent neural networks), a facial expression recognition model (e.g., ResNet-based CNN), and a text emotion classification model (e.g., Transformer-based large language model), outputting emotion labels (e.g., stress, relaxation, hurry, etc.) and emotion scores (e.g., stress level 0.85, relaxation level 0.12, etc., as real values from 0 to 1) for each modality. The ordering unit integrates these emotion estimation results, combines them with time, context, and past emotion history, and executes an ordering interface optimization algorithm (e.g., minimizing choices when stress is high, increasing customization options when relaxation is high, presenting one-touch ordering screens when hurry is high, etc., using rule-based branching). For example, input examples include “Voice: tense tone, Facial image: frown, Text: work is tough” or “Voice: calm tone, Facial image: smile, Text: had a relaxing day,” and output examples include “Emotion: stress, Ordering UI: minimal choices,” “Emotion: relaxation, Ordering UI: detailed customization,” “Emotion: hurry, Ordering UI: one-touch ordering.” The ordering unit automatically adjusts the number of items, display order, and input method (e.g., voice input, template selection, automatic completion from history, etc.) of the ordering screen according to the estimated emotions, optimizing the user experience. For training the emotion estimation model, annotated emotion datasets are used for each modality, applying cross-entropy loss or multitask loss as the loss function, and optimizing weights using gradient descent. Furthermore, data augmentation such as pitch shifting for audio, horizontal flipping for images, and synonym replacement for text is performed to improve generalization performance. Unlike conventional human-based subjective ordering screen adjustment or uniform ordering procedures, the ordering unit implements computer-specific, non-conventional procedures such as multimodal emotion estimation, rule-based branching, and automatic UI optimization in high-dimensional feature space, thereby achieving significant technical effects such as improved ordering efficiency, user experience, prevention of erroneous operations, and order completion rate. Specific application fields include personalized ingredient ordering applications, stress management support ordering systems, and ingredient ordering platforms for corporate welfare.

The ordering unit can analyze a user's past order history during ordering and select the optimal ordering method. For example, the ordering unit automatically selects frequently ordered ingredients based on the user's past order history. Additionally, the ordering unit can set a regular ordering schedule based on the user's past order history. Furthermore, the ordering unit can prioritize the ordering of specific ingredients based on the user's past order history. By analyzing the user's past order history, the ordering unit can select the optimal ordering method. Specifically, the ordering unit obtains more than one year of past order history data for each user (e.g., order date, ingredient name, quantity, order destination, order frequency, allergy information, inventory status, etc., as structured data) from the database. The ordering unit applies frequency analysis algorithms (e.g., counting occurrences of each ingredient, extracting time-series patterns), periodicity detection algorithms (e.g., autocorrelation analysis, periodicity scoring), and priority evaluation algorithms (e.g., calculating scores based on recent consumption, inventory status, user preferences, and relevance to health conditions) to these history data. The ordering unit automatically adds frequently ordered ingredients to the candidate list, and if regular ordering patterns are detected, generates automatic reminders or regular ordering schedules (e.g., ordering milk and eggs every Monday). For example, input examples include “Order history for the past 12 months: milk 20 times, eggs 18 times, tomatoes 5 times,” “Recent inventory: milk x, eggs ○,” and output examples include “Automatic order candidate: milk, Order frequency: once a week, Priority: high.” The ordering unit monitors the user's ordering trends and inventory fluctuations in real time, dynamically updating order candidates and schedules. Furthermore, the ordering unit can accumulate user feedback and order completion rates as training data, and optimize the parameters of the ordering algorithm using reinforcement learning. Unlike conventional human-based manual ordering or simple history reference, the ordering unit implements computer-specific, non-conventional procedures such as high-dimensional history analysis, automatic priority assignment, and adaptive order scheduling, thereby achieving significant technical effects such as improved ordering efficiency, inventory management accuracy, user satisfaction, and prevention of erroneous orders. Specific application fields include order support systems linked to online supermarkets, ingredient delivery services, and automatic ordering platforms for corporate welfare.

The ordering unit can customize the means of ordering based on the user's current living conditions during ordering. For example, if the user is busy, the ordering unit provides a method for quick ordering. Additionally, if the user is relaxed, the ordering unit can provide detailed ordering options. Furthermore, if the user is traveling, the ordering unit can provide a method for ordering at the travel destination. By customizing the means of ordering based on the user's current living conditions, the ordering unit can provide more appropriate ordering methods. Specifically, the ordering unit receives user profile data (e.g., current activity status flags such as “busy,” “relaxed,” “traveling,” schedule information, location information, device usage status, etc., as structured data) as input. The ordering unit implements an ordering means selection algorithm (e.g., one-touch ordering screen when busy, detailed customization screen when relaxed, local online supermarket linkage screen when traveling, etc.) in conjunction with profile information. The ordering unit automatically adjusts the number of items, display order, and input method (e.g., voice input, automatic completion from history, support for local languages, etc.) of the ordering screen according to the user's status, optimizing the number of steps and time required to complete the order. For example, input examples include “User status: busy, Current location: home, Device: smartphone,” “User status: relaxed, Current location: home, Device: tablet,” “User status: traveling, Current location: Osaka, Device: smartphone,” and output examples include “Ordering UI: one-touch ordering,” “Ordering UI: detailed customization,” “Ordering UI: local online supermarket linkage.” The ordering unit detects changes in the user's living conditions and device usage status in real time, dynamically updating the ordering means selection rules to maintain an optimal ordering experience at all times. Furthermore, the ordering unit can accumulate user feedback and order completion rates as training data, and optimize the parameters of the ordering means selection algorithm using reinforcement learning. Unlike conventional human-based uniform ordering means or manual selection, the ordering unit implements computer-specific, non-conventional procedures such as high-dimensional profile analysis, automatic means selection, and adaptive UI optimization, thereby achieving significant technical effects such as improved ordering efficiency, user satisfaction, and order completion rate. Specific application fields include personalized ingredient ordering applications, ingredient ordering services for travelers, and corporate welfare support systems.

The ordering unit can estimate a user's emotions and determine the priority of orders based on the estimated emotions. For example, if the user feels stressed, the ordering unit prioritizes the ordering of ingredients with relaxation effects. Additionally, if the user is relaxed, the ordering unit can prioritize the ordering of healthy ingredients. Furthermore, if the user is in a hurry, the ordering unit can prioritize the ordering of ingredients that can be cooked quickly. By determining the priority of orders based on the user's emotions, the ordering unit can preferentially order more appropriate ingredients. Emotion estimation is realized using emotion engines or generative AI, such as text generation AI (e.g., LLMs) or multimodal generative AI, but is not limited to these examples. Specifically, the ordering unit receives various input data for emotion estimation, such as voice data (e.g., 1-minute audio waveform, 16 kHz sampling), facial image data (e.g., 640×480 pixel RGB images), and text data (e.g., SNS posts or diary entries, up to 512 tokens). The ordering unit applies a voice emotion recognition model (e.g., CNN+RNN), a facial expression recognition model (e.g., ResNet-based CNN), and a text emotion classification model (e.g., Transformer-based large language model), outputting emotion labels (e.g., stress, relaxation, hurry, etc.) and emotion scores (e.g., stress level 0.8, relaxation level 0.1, etc.). The ordering unit calculates priority scores for candidate ingredients (e.g., degree of relaxation effect, healthiness, cooking time, etc.) according to the estimated emotions, and implements a priority determination algorithm (e.g., weighting based on emotion scores, branching by threshold judgment). For example, input examples include “Voice: tense tone, Facial image: frown, Text: work is tough” or “Voice: calm tone, Facial image: smile, Text: had a relaxing day,” and output examples include “Emotion: stress, Priority order: ingredients with relaxation effects,” “Emotion: relaxation, Priority order: healthy ingredients,” “Emotion: hurry, Priority order: quick-cooking ingredients.” The ordering unit automatically adjusts the order of candidate lists and the display order of the ordering screen according to the priority, realizing ingredient selection and ordering optimized for the user's state. For training the emotion estimation model, annotated emotion datasets are used for each modality, applying cross-entropy loss or multitask loss, and optimizing weights using gradient descent. Unlike conventional human-based subjective priority assignment or uniform ingredient selection, the ordering unit implements computer-specific, non-conventional procedures such as multimodal emotion estimation, automatic priority determination, and adaptive ingredient selection, thereby achieving significant technical effects such as improved ordering efficiency, relevance, and user experience. Specific application fields include stress management support ordering systems, personalized ingredient ordering services, and health management support platforms.

The ordering unit can select the optimal ordering method by considering the user's geographic location information during ordering. For example, if the user is in a specific region, the ordering unit prioritizes ordering from online supermarkets in that region. Additionally, if the user is traveling, the ordering unit can order from online supermarkets at the travel destination. Furthermore, if the user is at a specific exercise facility, the ordering unit can order from online supermarkets near that facility. By considering the user's geographic location information, the ordering unit can select the optimal ordering method. Specifically, the ordering unit obtains the user's current location information (e.g., GPS coordinates, latitude and longitude data, location accuracy within 5 meters) in real time and links with a geographic information database (e.g., list of online supermarkets by region, delivery area information, store inventory information, etc.). The ordering unit implements a geographic information filtering algorithm (e.g., distance weighting, delivery availability determination, branching by facility type) that calculates priority scores for candidate online supermarkets (e.g., distance, delivery time, inventory status, price, etc.) in conjunction with location information. The ordering unit automatically displays optimal online supermarket candidates on the ordering screen based on priority scores, suppressing unnecessary choices. For example, input examples include “Current location: Chiyoda-ku, Tokyo, Facility: gym,” “Current location: Osaka City, traveling,” and output examples include “Order destination candidates: Online Supermarket A (5 minutes on foot), Online Supermarket B (30 minutes delivery).” The ordering unit detects user movement and location changes in real time, dynamically updating the online supermarket candidate list and ordering rules to maintain an optimal ordering experience at all times. Furthermore, the ordering unit learns the relationship between geographic information and order history using machine learning models (e.g., random forests, gradient boosting decision trees, etc.), calculates order destination prediction scores, and performs branching control at the threshold determination unit. Unlike conventional human-based manual location input or uniform order destination selection, the ordering unit implements computer-specific, non-conventional procedures such as real-time location information analysis, automatic priority assignment, and adaptive order destination selection, thereby achieving significant technical effects such as improved ordering efficiency, delivery optimization, and user satisfaction. Specific application fields include ingredient ordering applications for travelers, ordering systems linked to exercise facilities, and community-based online supermarket linkage services.

The ordering unit can analyze a user's social media activity during ordering and propose means of ordering. For example, the ordering unit orders related ingredients based on meal photographs shared by the user on social media. Additionally, the ordering unit can order healthy ingredients based on health-related posts shared by the user on social media. Furthermore, the ordering unit can order ingredients needed after exercise based on exercise records shared by the user on social media. By analyzing the user's social media activity, the ordering unit can propose more appropriate means of ordering. Specifically, with the user's permission, the ordering unit automatically obtains post data (e.g., text posts, images, shared exercise records, etc.) from major social media platforms via API. The ordering unit receives text data (e.g., post text up to 512 tokens), image data (e.g., 640×480 pixel JPEG images), and exercise record data (e.g., date, activity type, distance, calories burned, etc., as structured data) as input, and applies a text analysis model (e.g., Transformer-based large language model), image analysis model (e.g., CNN for meal image classification), and exercise record analysis model (e.g., time-series clustering algorithm). The ordering unit automatically extracts dietary trends (e.g., high fat, low carbohydrate, vegetable-focused, etc.), health conditions (e.g., poor physical condition, fatigue, good condition, etc.), and exercise performance indicators (e.g., running distance, calories burned) from post content, and calculates priority scores for candidate ingredients and ordering timing. For example, input examples include “Post: ran 10 km today, Image: salad and steak photo,” “Post: feeling tired lately, Image: fast food photo,” and output examples include “Order candidate: protein supplement ingredients after exercise, Order timing: after exercise,” “Order candidate: health recovery ingredients, Order timing: immediate.” The ordering unit detects changes in social media activity in real time, dynamically updating ordering means and candidate ingredient lists to always provide the latest and most relevant ordering proposals. Furthermore, the ordering unit accumulates user posting trends and feedback as training data, continuously optimizing the parameters of the analysis model. Unlike conventional human-based manual post analysis or uniform ordering means, the ordering unit implements computer-specific, non-conventional procedures such as multimodal post analysis, automatic priority assignment, and adaptive ordering proposals, thereby achieving significant technical effects such as improved ordering efficiency, relevance, and user experience. Specific application fields include SNS-linked ingredient ordering applications, personalized health support services, and meal and exercise trend analysis platforms.

The recommendation unit can estimate a user's emotions and adjust the manner of presenting dining-out information based on the estimated emotions. For example, if the user is nervous, the recommendation unit introduces restaurants where the user can relax. Additionally, if the user is relaxed, the recommendation unit can introduce restaurants with healthy menus. Furthermore, if the user is in a hurry, the recommendation unit can introduce restaurants where meals can be served quickly. By adjusting the manner of presenting dining-out information based on the user's emotions, the recommendation unit can provide more appropriate dining-out information. Emotion estimation is realized using emotion engines or generative AI, such as text generation AI (e.g., LLMs) or multimodal generative AI, but is not limited to these examples. Specifically, the recommendation unit receives various input data for emotion estimation, such as voice data (e.g., 1-minute audio waveform, 16 kHz sampling, monaural PCM), facial image data (e.g., 640×480 pixel RGB images), and text data (e.g., SNS posts or diary entries, up to 512 tokens). The recommendation unit applies a voice emotion recognition model (e.g., hybrid configuration of convolutional neural networks and recurrent neural networks), a facial expression recognition model (e.g., ResNet-based CNN), and a text emotion classification model (e.g., Transformer-based large language model), outputting emotion labels (e.g., nervous, relaxed, hurried, etc.) and emotion scores (e.g., nervousness 0.75, relaxation 0.15, etc., as real values from 0 to 1) for each modality. The recommendation unit integrates these emotion estimation results, combines them with time, context, and past emotion history, and executes a recommendation expression optimization algorithm (e.g., prioritizing restaurants with quiet atmosphere, lighting, and BGM information when nervousness is high; presenting healthy menu and nutrient information in detail when relaxation is high; emphasizing waiting time, serving speed, and takeout availability when hurried, etc., using rule-based branching). For example, input examples include “Voice: fast and high-pitched, Facial image: frown, Text: busy at work” or “Voice: calm tone, Facial image: smile, Text: had a relaxing day,” and output examples include “Emotion: nervous, Recommendation expression: quiet cafe with lighting information,” “Emotion: relaxed, Recommendation expression: detailed healthy menu information,” “Emotion: hurried, Recommendation expression: emphasis on serving time and takeout availability.” The recommendation unit automatically adjusts the number of items, display order, color scheme, map UI, and filtering conditions of the recommendation screen, optimizing the user experience. For training the emotion estimation model, annotated emotion datasets are used for each modality, applying cross-entropy loss or multitask loss as the loss function, and optimizing weights using gradient descent. Furthermore, data augmentation such as pitch shifting for audio, horizontal flipping for images, and synonym replacement for text is performed to improve generalization performance. Unlike conventional human-based subjective recommendation adjustment or uniform dining-out information presentation, the recommendation unit implements computer-specific, non-conventional procedures such as multimodal emotion estimation, rule-based branching, and automatic recommendation optimization in high-dimensional feature space, thereby achieving significant technical effects such as improved recommendation accuracy, user experience, and information transmission efficiency. Specific application fields include personalized dining-out recommendation applications, stress management support restaurant recommendation services, and patient dining-out guidance support tools for medical institutions.

The recommendation unit can refer to a user's past dining-out history when presenting dining-out information and provide optimal recommendations. For example, the recommendation unit introduces related restaurants based on restaurants the user has visited in the past. Additionally, the recommendation unit can introduce restaurants with preferred menus based on the user's past dining-out history. Furthermore, the recommendation unit can analyze the user's past dining-out history and introduce restaurants with the highest satisfaction. By referring to the user's past dining-out history, the recommendation unit can provide more appropriate dining-out information. Specifically, the recommendation unit obtains more than one year of past dining-out history data for each user (e.g., visit date, restaurant name, ordered menu, satisfaction score, length of stay, payment amount, review comments, etc., as structured data) from the database. The recommendation unit applies similar restaurant extraction algorithms (e.g., cosine similarity calculation of restaurant attribute vectors, menu feature clustering), preference estimation algorithms (e.g., natural language analysis of order frequency, satisfaction, and review content), and satisfaction prediction models (e.g., satisfaction score regression using gradient boosting decision trees) to these history data. The recommendation unit extracts features of restaurants frequently visited or highly rated by the user, and adds similar new restaurants or unvisited establishments to the recommendation candidate list with priority. For example, input examples include “Dining-out history for the past 12 months: Restaurant A (Japanese cuisine, satisfaction 4.8), Restaurant B (Italian, satisfaction 4.2), Ordered menu: salad, pasta, fish dishes,” and output examples include “Recommendation candidates: Restaurant C (Japanese cuisine, abundant salads), Restaurant D (Italian, seafood menu).” The recommendation unit monitors changes in user preferences and satisfaction in real time, dynamically updating the recommendation candidate list and priority scores. Furthermore, the recommendation unit can accumulate user feedback and post-recommendation visit history as training data, and optimize the parameters of the recommendation algorithm using reinforcement learning. Unlike conventional human-based manual history reference or simple ranking presentation, the recommendation unit implements computer-specific, non-conventional procedures such as high-dimensional history analysis, automatic similarity evaluation, and adaptive recommendation scheduling, thereby achieving significant technical effects such as improved recommendation efficiency, satisfaction, user experience, and discovery rate. Specific application fields include personalized dining-out recommendation services, restaurant recommendation applications linked to meal history, and dining-out support platforms for corporate welfare.

The recommendation unit can estimate a user's emotions and determine the priority of dining-out information based on the estimated emotions. For example, if the user feels stressed, the recommendation unit prioritizes the introduction of restaurants where the user can relax. Additionally, if the user is relaxed, the recommendation unit can prioritize the introduction of restaurants with healthy menus. Furthermore, if the user is in a hurry, the recommendation unit can prioritize the introduction of restaurants where meals can be served quickly. By determining the priority of dining-out information based on the user's emotions, the recommendation unit can provide more appropriate dining-out information. Emotion estimation is realized using emotion engines or generative AI, such as text generation AI (e.g., LLMs) or multimodal generative AI, but is not limited to these examples. Specifically, the recommendation unit receives various input data for emotion estimation, such as voice data (e.g., 1-minute audio waveform, 16 kHz sampling), facial image data (e.g., 640×480 pixel RGB images), and text data (e.g., SNS posts or diary entries, up to 512 tokens). The recommendation unit applies a voice emotion recognition model (e.g., CNN+RNN), a facial expression recognition model (e.g., ResNet-based CNN), and a text emotion classification model (e.g., Transformer-based large language model), outputting emotion labels (e.g., stress, relaxation, hurry, etc.) and emotion scores (e.g., stress level 0.8, relaxation level 0.1, etc.). The recommendation unit calculates priority scores for candidate restaurants (e.g., degree of relaxation, healthiness, serving speed, etc.) according to the estimated emotions, and implements a priority determination algorithm (e.g., weighting based on emotion scores, branching by threshold judgment). For example, input examples include “Voice: tense tone, Facial image: frown, Text: work is tough” or “Voice: calm tone, Facial image: smile, Text: had a relaxing day,” and output examples include “Emotion: stress, Priority recommendation: restaurant with relaxation effect,” “Emotion: relaxation, Priority recommendation: healthy menu,” “Emotion: hurry, Priority recommendation: quick meal restaurant.” The recommendation unit automatically adjusts the order of candidate lists and the display order of the recommendation screen according to the priority, realizing dining-out information provision optimized for the user's state. For training the emotion estimation model, annotated emotion datasets are used for each modality, applying cross-entropy loss or multitask loss, and optimizing weights using gradient descent. Unlike conventional human-based subjective priority assignment or uniform restaurant selection, the recommendation unit implements computer-specific, non-conventional procedures such as multimodal emotion estimation, automatic priority determination, and adaptive recommendation control, thereby achieving significant technical effects such as improved recommendation efficiency, relevance, and user experience. Specific application fields include stress management support dining-out recommendation systems, personalized restaurant recommendation services, and health management support platforms.

The recommendation unit can provide optimal recommendations by considering the user's geographic location information when presenting dining-out information. For example, if the user is in a specific region, the recommendation unit prioritizes the introduction of restaurants in that region. Additionally, if the user is traveling, the recommendation unit can introduce restaurants at the travel destination. Furthermore, if the user is at a specific exercise facility, the recommendation unit can introduce restaurants near that facility. By considering the user's geographic location information, the recommendation unit can provide more appropriate dining-out information. Specifically, the recommendation unit obtains the user's current location information (e.g., GPS coordinates, latitude and longitude data, location accuracy within 5 meters) in real time and links with a geographic information database (e.g., list of restaurants by region, business hours, congestion status, availability of healthy menus, facility information, etc.). The recommendation unit implements a geographic information filtering algorithm (e.g., distance weighting, congestion filtering, branching by facility type) that calculates priority scores for candidate restaurants (e.g., distance, congestion level, availability of healthy menus, price range, serving speed, etc.) in conjunction with location information. The recommendation unit automatically displays optimal restaurant candidates on the recommendation screen based on priority scores, suppressing unnecessary choices. For example, input examples include “Current location: Chiyoda-ku, Tokyo, Facility: gym,” “Current location: Osaka City, traveling,” and output examples include “Recommendation destination candidates: Restaurant A (5 minutes on foot), Restaurant B (low congestion, healthy menu).” The recommendation unit detects user movement and location changes in real time, dynamically updating the restaurant candidate list and recommendation rules to maintain an optimal recommendation experience at all times. Furthermore, the recommendation unit learns the relationship between geographic information and dining-out history using machine learning models (e.g., random forests, gradient boosting decision trees, etc.), calculates recommendation destination prediction scores, and performs branching control at the threshold determination unit. Unlike conventional human-based manual location input or uniform restaurant selection, the recommendation unit implements computer-specific, non-conventional procedures such as real-time location information analysis, automatic priority assignment, and adaptive recommendation destination selection, thereby achieving significant technical effects such as improved recommendation efficiency, convenience, and user satisfaction. Specific application fields include restaurant recommendation applications for travelers, dining-out support systems linked to exercise facilities, and community-based healthy dining-out recommendation services.

The system according to the embodiment is not limited to the examples described above and can be variously modified as follows, for example. Specifically, the system can be applied to a wide range of fields beyond health, exercise, and meal management, such as sleep management, stress monitoring, medication management, rehabilitation support, lifestyle disease prevention, mental healthcare, senior monitoring, child growth recording, corporate health management support, performance optimization for sports teams, health support for travelers, and health information collection, analysis, and proposal during disasters. The system can also flexibly change the types of AI models and algorithm configurations, for example, by extending image recognition models to medical image diagnosis or food recognition, applying time-series analysis models to long-term trend analysis of sleep, heart rate, blood pressure, and blood glucose, or applying natural language processing models to doctor-patient communication or health consultation chatbots. Furthermore, various hardware devices can be combined for data collection, such as wearable devices, smartphones, IoT home appliances, medical devices, environmental sensors, and smart home-linked devices. In addition, system configurations and operational forms can include various variations such as cloud integration, edge AI processing, distributed databases, enhanced privacy protection, multi-user and multi-location support, and internationalization. As a result, the system can provide not only a simple health management application but also broad technical effects and social value, such as promoting overall societal health, reducing medical costs, improving quality of life, industrial applications, disaster response, and educational support.

The analysis unit can estimate a user's emotions and determine the priority of analysis based on the estimated emotions. For example, if the user feels stressed, the analysis unit prioritizes the analysis of health information related to stress reduction. Additionally, if the user is relaxed, the analysis unit can prioritize the analysis of information related to exercise performance. Furthermore, if the user is in a hurry, the analysis unit can prioritize the analysis of information that can be analyzed in a short time. By determining the priority of analysis based on the user's emotions, the analysis unit can provide more appropriate analysis results. Specifically, the analysis unit receives various input data for emotion estimation, such as voice data (e.g., 1-minute audio waveform, 16 kHz sampling), facial image data (e.g., 640×480 pixel RGB images), and text data (e.g., SNS posts or diary entries, up to 512 tokens). The analysis unit applies a voice emotion recognition model (e.g., hybrid configuration of convolutional neural networks and recurrent neural networks), a facial expression recognition model (e.g., ResNet-based CNN), and a text emotion classification model (e.g., Transformer-based large language model), outputting emotion labels (e.g., stress, relaxation, hurry, etc.) and emotion scores (e.g., stress level 0.8, relaxation level 0.1, hurry level 0.7, etc., as real values from 0 to 1) for each modality. The analysis unit integrates these emotion estimation results, combines them with time, context, and past emotion history, and executes an analysis priority determination algorithm (e.g., weighting based on emotion scores, branching by threshold judgment). For example, input examples include “Voice: tense tone, Facial image: frown, Text: work is tough” or “Voice: calm tone, Facial image: smile, Text: exercised today,” and output examples include “Emotion: stress, Priority analysis: stress reduction-related information,” “Emotion: relaxation, Priority analysis: exercise performance information,” “Emotion: hurry, Priority analysis: information that can be analyzed quickly.” The analysis unit automatically adjusts the order and level of detail of analysis target data (e.g., health indicator vectors, exercise time-series arrays, meal image feature vectors, etc.) according to the priority, realizing analysis optimized for the user's state. For training the emotion estimation model, annotated emotion datasets are used for each modality, applying cross-entropy loss or multitask loss, and optimizing weights using gradient descent. Furthermore, data augmentation such as pitch shifting for audio, horizontal flipping for images, and synonym replacement for text is performed to improve generalization performance. Unlike conventional human-based subjective priority assignment or uniform analysis order settings, the analysis unit implements computer-specific, non-conventional procedures such as multimodal emotion estimation, automatic priority determination, and adaptive analysis order control, thereby achieving significant technical effects such as improved analysis efficiency, relevance, and user experience. Specific application fields include stress management support health analysis systems, personalized health report generation services, and patient state analysis platforms for medical institutions.

The proposal unit is capable of estimating a user's emotions and adjusting the timing of proposals based on the estimated emotions. For example, if the user is feeling stressed, proposals are made during periods when the user is relaxed. If the user is relaxed, proposals may be made after exercise. Furthermore, if the user is in a hurry, information that can be proposed in a short time is prioritized and provided. In this way, the proposal unit can provide more appropriate proposals by adjusting the timing of proposals based on the user's emotions. Specifically, the proposal unit receives diverse input data for emotion estimation, such as voice data (e.g., one-minute audio waveform, 16 kHz sampling), facial image data (e.g., 640×480 pixel RGB images), and text data (e.g., SNS posts or diary entries, up to 512 tokens). The proposal unit applies a voice emotion recognition model (e.g., a hybrid configuration of convolutional neural networks and recurrent neural networks), a facial expression recognition model (e.g., ResNet-based CNN), and a text emotion classification model (e.g., Transformer-based large language model), and outputs emotion labels (e.g., stress, relaxation, hurry, etc.) and emotion scores (e.g., stress level 0.7, relaxation level 0.2, hurry level 0.6, etc.) for each modality. The proposal unit integrates these emotion estimation results and combines them with time, situation, and past emotion history to execute a proposal timing optimization algorithm (e.g., rule-based branching or reinforcement learning model combining emotion scores with the user's life rhythm, exercise history, and calendar information). For example, input examples include “Voice: tense tone, facial image: frown, text: work is tough” or “Voice: calm tone, facial image: smile, text: exercised today,” and output examples include “Emotion: stress, proposal timing: evening relaxation time,” “Emotion: relaxation, proposal timing: after exercise,” “Emotion: hurry, proposal timing: immediate, summary only.” The proposal unit links the estimated emotions with the user's behavioral history and schedule information, automatically adjusts the timing and detail level of proposal notifications, and maximizes user receptivity and behavior change rate. For training the emotion estimation models, annotated emotion datasets are used for each modality, cross-entropy loss and multitask loss are applied, and weights are optimized by gradient descent. Unlike conventional subjective timing adjustments or uniform proposal notifications by humans, the proposal unit implements computer-specific, non-conventional procedures such as multimodal emotion estimation, automatic timing optimization, and adaptive proposal notification control, thereby achieving significant technical effects such as improved proposal acceptance rate, user experience, and behavior change support. Specific application fields include personalized health proposal applications, stress management support services, and exercise and dietary habit improvement support systems.

The ordering unit is capable of estimating a user's emotions and adjusting the level of detail of orders based on the estimated emotions. For example, if the user is feeling stressed, a simple ordering interface is provided. If the user is relaxed, detailed ordering options may be provided. Furthermore, if the user is in a hurry, a method for quick ordering may be provided. In this way, the ordering unit can provide a more appropriate ordering method by adjusting the level of detail of orders based on the user's emotions. Specifically, the ordering unit receives diverse input data for emotion estimation, such as voice data (e.g., one-minute audio waveform, 16 kHz sampling), facial image data (e.g., 640×480 pixel RGB images), and text data (e.g., SNS posts or diary entries, up to 512 tokens). The ordering unit applies a voice emotion recognition model (e.g., a hybrid configuration of convolutional neural networks and recurrent neural networks), a facial expression recognition model (e.g., ResNet-based CNN), and a text emotion classification model (e.g., Transformer-based large language model), and outputs emotion labels (e.g., stress, relaxation, hurry, etc.) and emotion scores (e.g., stress level 0.85, relaxation level 0.12, hurry level 0.7, etc.) for each modality. The ordering unit integrates these emotion estimation results and combines them with time, situation, and past emotion history to execute an ordering interface optimization algorithm (e.g., rule-based branching such as minimizing choices when stress level is high, increasing customization items when relaxation level is high, and presenting one-touch ordering screens when hurry level is high). For example, input examples include “Voice: tense tone, facial image: frown, text: work is tough” or “Voice: calm tone, facial image: smile, text: had a relaxing day today,” and output examples include “Emotion: stress, ordering UI: minimal choices,” “Emotion: relaxation, ordering UI: detailed customization,” “Emotion: hurry, ordering UI: one-touch order.” The ordering unit automatically adjusts the number of items, display order, and input methods (e.g., voice input, template selection, automatic completion from history, etc.) of the ordering screen according to the estimated emotions to optimize the user experience. For training the emotion estimation models, annotated emotion datasets are used for each modality, cross-entropy loss and multitask loss are applied as loss functions, and weights are optimized by gradient descent. Furthermore, data augmentation such as pitch shifting for voice, horizontal flipping for images, and synonym replacement for text is performed to improve generalization performance. Unlike conventional subjective ordering screen adjustments or uniform ordering procedures by humans, the ordering unit implements computer-specific, non-conventional procedures such as multimodal emotion estimation in high-dimensional feature space, rule-based branching, and automatic UI optimization, thereby achieving significant technical effects such as improved ordering efficiency, user experience, prevention of erroneous operations, and order completion rate. Specific application fields include personalized ingredient ordering applications, stress management support ordering systems, and ingredient ordering platforms for corporate welfare.

The recommendation unit is capable of estimating a user's emotions and adjusting the timing of providing dining-out information based on the estimated emotions. For example, if the user is feeling stressed, dining-out information is provided during periods when the user can relax. If the user is relaxed, dining-out information may be provided before meals. Furthermore, if the user is in a hurry, information about restaurants where meals can be served quickly may be prioritized and provided. In this way, the recommendation unit can provide more appropriate dining-out information by adjusting the timing of providing dining-out information based on the user's emotions. Specifically, the recommendation unit receives diverse input data for emotion estimation, such as voice data (e.g., one-minute audio waveform, 16 kHz sampling), facial image data (e.g., 640×480 pixel RGB images), and text data (e.g., SNS posts or diary entries, up to 512 tokens). The recommendation unit applies a voice emotion recognition model (e.g., a hybrid configuration of convolutional neural networks and recurrent neural networks), a facial expression recognition model (e.g., ResNet-based CNN), and a text emotion classification model (e.g., Transformer-based large language model), and outputs emotion labels (e.g., stress, relaxation, hurry, etc.) and emotion scores (e.g., stress level 0.7, relaxation level 0.2, hurry level 0.6, etc.) for each modality. The recommendation unit integrates these emotion estimation results and combines them with time, situation, and past emotion history to execute a recommendation timing optimization algorithm (e.g., rule-based branching or reinforcement learning model combining emotion scores with the user's life rhythm, meal history, and calendar information). For example, input examples include “Voice: tense tone, facial image: frown, text: work is tough” or “Voice: calm tone, facial image: smile, text: had a relaxing day today,” and output examples include “Emotion: stress, recommendation timing: evening relaxation time,” “Emotion: relaxation, recommendation timing: before meals,” “Emotion: hurry, recommendation timing: immediate, prioritize quick restaurants.” The recommendation unit links the estimated emotions with the user's behavioral history and schedule information, automatically adjusts the timing and detail level of recommendation notifications, and maximizes user receptivity and visit rate. For training the emotion estimation models, annotated emotion datasets are used for each modality, cross-entropy loss and multitask loss are applied, and weights are optimized by gradient descent. Unlike conventional subjective timing adjustments or uniform recommendation notifications by humans, the recommendation unit implements computer-specific, non-conventional procedures such as multimodal emotion estimation, automatic timing optimization, and adaptive recommendation notification control, thereby achieving significant technical effects such as improved recommendation acceptance rate, user experience, and support for dining-out behavior. Specific application fields include personalized dining-out recommendation applications, stress management support restaurant recommendation services, and meal behavior change support systems.

The collection unit is capable of estimating a user's emotions and determining the types of health and exercise information to be collected based on the estimated emotions. For example, if the user is feeling stressed, information related to stress reduction is prioritized for collection. If the user is relaxed, information related to exercise performance may be prioritized for collection. Furthermore, if the user is in a hurry, information that can be collected in a short time may be prioritized for collection. In this way, the collection unit can collect more appropriate information by determining the types of information to be collected based on the user's emotions. Specifically, the collection unit receives diverse input data for emotion estimation, such as voice data (e.g., one-minute audio waveform, 16 kHz sampling), facial image data (e.g., 640×480 pixel RGB images), and text data (e.g., SNS posts or diary entries, up to 512 tokens). The collection unit applies a voice emotion recognition model (e.g., a hybrid configuration of convolutional neural networks and recurrent neural networks), a facial expression recognition model (e.g., ResNet-based CNN), and a text emotion classification model (e.g., Transformer-based large language model), and outputs emotion labels (e.g., stress, relaxation, hurry, etc.) and emotion scores (e.g., stress level 0.8, relaxation level 0.1, hurry level 0.7, etc.) for each modality. The collection unit integrates these emotion estimation results and combines them with time, situation, and past emotion history to execute a priority determination algorithm for target data to be collected (e.g., weighting based on emotion scores, branching by threshold judgment). For example, input examples include “Voice: tense tone, facial image: frown, text: work is tough” or “Voice: calm tone, facial image: smile, text: exercised today,” and output examples include “Emotion: stress, priority collection: stress reduction-related information,” “Emotion: relaxation, priority collection: exercise performance information,” “Emotion: hurry, priority collection: information that can be collected quickly.” The collection unit automatically adjusts the collection frequency, timing, and devices (e.g., wearable devices, smartphone apps, etc.) according to the priority, thereby realizing information collection optimized for the user's condition. For training the emotion estimation models, annotated emotion datasets are used for each modality, cross-entropy loss and multitask loss are applied, and weights are optimized by gradient descent. Unlike conventional subjective prioritization or uniform information collection by humans, the collection unit implements computer-specific, non-conventional procedures such as multimodal emotion estimation, automatic priority determination, and adaptive information collection, thereby achieving significant technical effects such as improved collection efficiency, relevance, and user experience. Specific application fields include stress management support applications, personalized health management services, and mental health monitoring systems.

The analysis unit is capable of analyzing a user's past health checkup results and adjusting the level of detail of analysis based on specific health indicators. For example, if past health checkup results indicate a tendency for high blood pressure, information related to blood pressure is analyzed in detail. If past health checkup results indicate a tendency for high blood glucose, information related to blood glucose may be analyzed in detail. Furthermore, if past health checkup results indicate a tendency for high cholesterol, information related to cholesterol may be analyzed in detail. In this way, the analysis unit can provide more appropriate analysis results by adjusting the level of detail of analysis based on past health checkup results. Specifically, the analysis unit acquires past health checkup result data for each user spanning more than one year (e.g., numerical vectors with dates for blood pressure, blood glucose, cholesterol, BMI, liver function, kidney function, etc.) from a database. The analysis unit applies anomaly detection algorithms (e.g., time-series trend analysis, calculation of anomaly scores using moving averages and standard deviations), risk assessment models (e.g., health risk score regression using random forests or gradient boosting decision trees), and importance evaluation algorithms (e.g., weighting based on physician instructions, past anomaly detection history, and user goals) to these historical data. For health indicators with high importance scores, detailed analysis (e.g., trend graphs for the past year, anomaly warnings, lifestyle advice, factor decomposition, annotation, etc.) is performed, and for items with low importance, summary analysis (e.g., presenting only averages and medians, simple graphs, etc.) is performed. For example, input examples include “Blood pressure: 140/90 mmHg (importance 0.95), blood glucose: 110 mg/dL (importance 0.8), cholesterol: 220 mg/dL (importance 0.9),” and output examples include “Blood pressure: trend graph for the past year, anomaly warning, lifestyle advice; blood glucose: detailed trend analysis; cholesterol: factor decomposition and recommended improvements.” The analysis unit dynamically adjusts the weighting of importance scores according to the user's goals and instructions from medical institutions to optimize the level of detail of analysis. Importance evaluation models use machine learning models such as random forests and gradient boosting decision trees, and parameters are optimized using past analysis results and user feedback as training data. Unlike conventional uniform analysis or detail level settings based on empirical rules by humans, the analysis unit implements computer-specific, non-conventional procedures such as high-dimensional historical analysis, automatic importance evaluation, and detail level optimization, thereby achieving significant technical effects such as improved analysis efficiency, accuracy, and user satisfaction. Specific application fields include chronic disease management systems, personalized health report generation services, and patient monitoring tools for medical institutions.

The proposal unit is capable of analyzing a user's past meal history and adjusting the level of detail of proposals based on specific dietary trends. For example, if past meal history indicates a tendency for low vegetable intake, cooking recipes rich in vegetables are proposed in detail. If past meal history indicates a tendency for low protein intake, cooking recipes rich in protein may be proposed in detail. Furthermore, if past meal history indicates a tendency for high calorie intake, low-calorie cooking recipes may be proposed in detail. In this way, the proposal unit can provide more appropriate proposals by adjusting the level of detail of proposals based on past meal history. Specifically, the proposal unit acquires past meal history data for each user spanning more than one year (e.g., structured data including date, dish name, ingredients, nutrients, calories, intake amount, meal photographs, etc.) from a database. The proposal unit applies dietary trend analysis algorithms (e.g., time-series aggregation of nutrient intake, extraction of meal patterns by clustering), nutrient deficiency detection models (e.g., comparison with recommended intake, calculation of deficiency scores), and importance evaluation algorithms (e.g., weighting based on physician instructions, health goals, and past anomaly detection history) to these historical data. For dietary trends with high importance scores, detailed proposals (e.g., nutrient breakdown tables for vegetable-focused recipes, detailed cooking procedures, recommended intake timing, etc.) are generated, and for trends with low importance, summary proposals (e.g., only dish name and calories, simple ingredient lists, etc.) are generated. For example, input examples include “Average daily vegetable intake for the past month: 50 g/day (recommended 200 g/day), protein intake: 40 g/day (recommended 60 g/day), calorie intake: 2500 kcal/day (recommended 1800 kcal/day),” and output examples include “Recommended recipes: vegetable-rich soup (with detailed nutrient breakdown table), grilled chicken breast (protein-enhanced), low-calorie Japanese set meal.” The proposal unit dynamically adjusts the weighting of proposal detail level according to the user's goals and instructions from medical institutions to realize optimal recipe proposals. Importance evaluation models use machine learning models such as random forests and gradient boosting decision trees, and parameters are optimized using past proposal history and user feedback as training data. Unlike conventional uniform proposals or detail level settings based on empirical rules by humans, the proposal unit implements computer-specific, non-conventional procedures such as high-dimensional historical analysis, automatic importance evaluation, and detail level optimization, thereby achieving significant technical effects such as improved proposal efficiency, accuracy, and user satisfaction. Specific application fields include chronic disease management support applications, personalized nutrition guidance services, and meal proposal systems for medical institutions.

The ordering unit is capable of analyzing a user's past order history and determining the priority of orders based on specific ingredients. For example, ingredients that are frequently ordered in past order history are prioritized for ordering. If a specific ingredient is found to be lacking in past order history, that ingredient may be prioritized for ordering. Furthermore, if a specific ingredient is found to be in surplus in past order history, recipes that prioritize the use of that ingredient may be proposed. In this way, the ordering unit can provide a more appropriate ordering method by determining the priority of orders based on past order history. Specifically, the ordering unit acquires past order history data for each user spanning more than one year (e.g., structured data including order date, ingredient name, quantity, order source, order frequency, allergy information, inventory status, etc.) from a database. The ordering unit applies frequency analysis algorithms (e.g., aggregation of occurrence counts for each ingredient, extraction of time-series patterns), inventory-linked algorithms (e.g., management of inventory quantity, consumption, expiration date), and priority evaluation algorithms (e.g., calculation of relevance scores based on recent consumption, inventory status, user preferences, and health conditions) to these historical data. The ordering unit automatically adds frequently ordered or lacking ingredients to the candidate list and proposes recipes that prioritize the use of ingredients with surplus inventory. For example, input examples include “Order history for the past 12 months: milk 20 times, eggs 18 times, tomatoes 5 times,” “Recent inventory: milk x, eggs ○, tomatoes ⊚,” and output examples include “Automatic order candidate: milk, order frequency: once a week, priority: high,” “Priority use recipe: tomato consumption recipe.” The ordering unit monitors user ordering trends and inventory fluctuations in real time and dynamically updates order candidates and schedules. Furthermore, the ordering unit can accumulate user feedback and order completion rates as training data and optimize the parameters of the ordering algorithm using reinforcement learning and other methods. Unlike conventional manual ordering or simple history reference by humans, the ordering unit implements computer-specific, non-conventional procedures such as high-dimensional historical analysis, automatic priority assignment, and adaptive order scheduling, thereby achieving significant technical effects such as improved ordering efficiency, inventory management accuracy, user satisfaction, and prevention of erroneous orders. Specific application fields include order support systems linked to online supermarkets, ingredient delivery services, and automatic ordering platforms for corporate welfare.

The recommendation unit is capable of analyzing a user's past dining-out history and determining the priority of dining-out information based on specific restaurants. For example, restaurants that are frequently visited in past dining-out history are prioritized for recommendation. If a specific restaurant is found to be preferred in past dining-out history, that restaurant may be prioritized for recommendation. Furthermore, if a specific restaurant is found to be unsatisfactory in past dining-out history, that restaurant may be excluded from recommendations. In this way, the recommendation unit can provide more appropriate dining-out information by determining the priority of dining-out information based on past dining-out history. Specifically, the recommendation unit acquires past dining-out history data for each user spanning more than one year (e.g., structured data including visit date, restaurant name, ordered menu, satisfaction score, stay time, payment amount, review comments, etc.) from a database. The recommendation unit applies similar restaurant extraction algorithms (e.g., cosine similarity calculation of restaurant attribute vectors, menu feature clustering), preference estimation algorithms (e.g., natural language analysis of order frequency, satisfaction, and review content), and satisfaction prediction models (e.g., satisfaction score regression using gradient boosting decision trees) to these historical data. The recommendation unit extracts features of restaurants frequently visited or highly rated by the user and adds similar new or unvisited restaurants to the recommendation candidate list, while excluding unsatisfactory restaurants from the candidates. For example, input examples include “Dining-out history for the past 12 months: Restaurant A (Japanese cuisine, satisfaction 4.8), Restaurant B (Italian, satisfaction 4.2), Restaurant C (Chinese, satisfaction 2.1),” and output examples include “Recommendation candidates: Restaurant D (Japanese cuisine, rich in salads), Restaurant E (Italian, seafood menu).” The recommendation unit monitors changes in user preferences and satisfaction in real time and dynamically updates the recommendation candidate list and priority scores. Furthermore, the recommendation unit can accumulate user feedback and post-recommendation visit history as training data and optimize the parameters of the recommendation algorithm using reinforcement learning and other methods. Unlike conventional manual history reference or simple ranking presentation by humans, the recommendation unit implements computer-specific, non-conventional procedures such as high-dimensional historical analysis, automatic similarity evaluation, and adaptive recommendation scheduling, thereby achieving significant technical effects such as improved recommendation efficiency, satisfaction, user experience, and discovery rate of new restaurants. Specific application fields include personalized dining-out recommendation services, dining-out history-linked restaurant recommendation applications, and dining-out support platforms for corporate welfare.

The collection unit is capable of determining the types of information to be collected based on specific regions by taking into account the user's geographic location information. For example, if the user is in a high-altitude area, information related to health risks at high altitudes is prioritized for collection. If the user is at the seaside, information related to health risks at the seaside may be prioritized for collection. Furthermore, if the user is in an urban area, information related to health risks in urban areas may be prioritized for collection. In this way, the collection unit can collect more appropriate information by considering the user's geographic location information. Specifically, the collection unit acquires the user's current location information (e.g., GPS coordinates, latitude and longitude data, location accuracy within 5 meters) in real time and links with a geographic information database (e.g., climate data by region, environmental risk information, facility information, etc.). The collection unit implements a geographic information filtering algorithm that calculates priority scores for target data to be collected in conjunction with location information (e.g., weighting based on region-specific risks, branching by facility type), such as oxygen saturation and heart rate in high-altitude areas, UV index and salt intake at the seaside, and air pollution index and allergen information in urban areas. The collection unit automatically selects collection frequency, timing, and devices based on priority scores and suppresses unnecessary data collection. For example, input examples include “Current location: high-altitude area at 2000 m elevation, temperature 5° C., facility: mountain trail,” “Current location: seaside, UV index 8, facility: beach,” “Current location: urban area, high PM2.5, facility: gym,” and output examples include “Priority collection: oxygen saturation and heart rate, collection frequency: every 30 minutes,” “Priority collection: UV index and salt intake, collection frequency: daytime,” “Priority collection: air pollution index and allergen information, collection frequency: before and after exercise.” The collection unit detects user movement and location changes in real time and dynamically updates geographic information filtering rules to maintain optimal information collection at all times. Furthermore, the collection unit learns the relationship between geographic information and health risks using machine learning models (e.g., random forests, gradient boosting decision trees, etc.), calculates risk prediction scores, and performs branching control at the threshold judgment unit. Unlike conventional manual location input or uniform information collection by humans, the collection unit implements computer-specific, non-conventional procedures such as real-time location information analysis, automatic priority assignment, and adaptive information collection, thereby achieving significant technical effects such as improved collection efficiency, relevance, and risk detection accuracy. Specific application fields include health management applications for travelers, environmental risk monitoring systems, and health support services linked to exercise facilities.

Below, the processing flow of Example of the Embodiment is briefly described. Specifically, the present system adopts a multilayer configuration in which each module—collection unit, analysis unit, proposal unit, ordering unit, and recommendation unit—operates in cooperation. First, the collection unit collects diverse data in real time, such as the user's health, exercise, dietary, emotional, location, and social media data, and the collected data are structured as high-dimensional vectors or time-series arrays. The system transfers the collected data to the analysis unit, which applies multiple machine learning algorithms (e.g., random forests, recurrent neural networks, Transformer-based large language models, etc.) for category determination, importance evaluation, emotion estimation, anomaly detection, trend analysis, and multifaceted analysis of user status, health risks, dietary trends, and emotional states. The system passes the analysis results to the proposal unit, which generates optimal recipes, nutritional guidance, and behavioral proposals considering the user's goals, health conditions, emotions, past history, and life rhythm. Based on the proposal content, the ordering unit performs automatic selection of necessary ingredients, generation of candidate order lists, online supermarket-linked ordering, inventory management, and optimization of order history. Furthermore, the recommendation unit searches menus of nearby restaurants and cafes based on the proposed recipes and the user's preferences, history, and location information, evaluates similarity, and prioritizes health-oriented dining-out information. The outputs of each unit are integrated and displayed on the user interface, and user feedback and behavioral history are accumulated as training data to continuously optimize the parameters of each algorithm using reinforcement learning and other methods. Unlike conventional manual data collection, analysis, proposal, ordering, and recommendation by humans, the present system implements computer-specific, non-conventional procedures such as high-dimensional data analysis, multimodal AI cooperation, and automatic optimization, thereby achieving significant technical effects such as improved health management efficiency, accuracy, user satisfaction, and support for behavior change. Specific application fields include personalized health management platforms, chronic disease management support systems, corporate health management support services, performance optimization systems for athletes, and health support applications for travelers.

Step 1: The collection unit collects health and exercise information. Health and exercise information includes heart rate, number of steps, calories burned, and meal content. The collection unit automatically collects data such as heart rate, number of steps, calories burned, and sleep time from wearable devices. It can also collect data such as health checkup results and daily meal photographs. For example, it collects health checkup results such as blood pressure, blood glucose, cholesterol, and meal photographs. Step 2: The analysis unit analyzes information collected by the collection unit. The analysis unit analyzes the collected data using statistical analysis and machine learning algorithms to grasp the user's health conditions, exercise status, and dietary trends. Step 3: The proposal unit proposes cooking recipes based on analysis results obtained by the analysis unit. The proposal unit proposes optimal cooking recipes based on the user's health conditions, exercise status, and dietary trends. For example, it proposes low-calorie cooking recipes for people on a diet, nutritionally balanced cooking recipes for people undergoing medical treatment, and high-protein cooking recipes for professional athletes. Step 4: The ordering unit places orders for ingredients based on recipes proposed by the proposal unit. The ordering unit automatically selects necessary ingredients based on the proposed cooking recipes and places orders with nearby online supermarkets. Step 5: The recommendation unit provides dining-out information based on recipes proposed by the proposal unit. The recommendation unit searches menus of nearby restaurants and cafes based on the proposed cooking recipes, selects dining-out information with menus similar to the relevant recipes, and provides recommendations. Specifically, the present system structures collected data as high-dimensional vectors or time-series arrays at each step, and the analysis unit applies multiple machine learning algorithms (e.g., random forests, recurrent neural networks, Transformer-based large language models, etc.) for category determination, importance evaluation, emotion estimation, anomaly detection, trend analysis, and multifaceted analysis of user status, health risks, dietary trends, and emotional states. The proposal unit generates optimal recipes, nutritional guidance, and behavioral proposals considering analysis results and the user's goals, health conditions, emotions, past history, and life rhythm; the ordering unit performs automatic selection of necessary ingredients, generation of candidate order lists, online supermarket-linked ordering, inventory management, and optimization of order history; and the recommendation unit searches menus of nearby restaurants and cafes based on the proposed recipes and the user's preferences, history, and location information, evaluates similarity, and prioritizes health-oriented dining-out information. The outputs of each unit are integrated and displayed on the user interface, and user feedback and behavioral history are accumulated as training data to continuously optimize the parameters of each algorithm using reinforcement learning and other methods. Unlike conventional manual data collection, analysis, proposal, ordering, and recommendation by humans, the present system implements computer-specific, non-conventional procedures such as high-dimensional data analysis, multimodal AI cooperation, and automatic optimization, thereby achieving significant technical effects such as improved health management efficiency, accuracy, user satisfaction, and support for behavior change. Specific application fields include personalized health management platforms, chronic disease management support systems, corporate health management support services, performance optimization systems for athletes, and health support applications for travelers.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the above-described collection unit, analysis unit, proposal unit, ordering unit, and recommendation unit is implemented by at least one of, for example, a smart device 14 and a data processing apparatus 12. For example, the collection unit collects health and exercise information using a camera 42 or a microphone 38B of the smart device 14, and collects data by a control unit 46A. The analysis unit is implemented, for example, by a specific processing unit 290 of the data processing apparatus 12 and analyzes the collected data. The proposal unit is implemented, for example, by the specific processing unit 290 of the data processing apparatus 12 and proposes cooking recipes based on analysis results. The ordering unit is implemented, for example, by the control unit 46A of the smart device 14 and places orders for ingredients based on the proposed recipes. The recommendation unit is implemented, for example, by the control unit 46A of the smart device 14 and provides dining-out information based on the proposed recipes. The correspondence between each unit and the device or control unit is not limited to the above examples and various modifications are possible.

### Second Embodiment

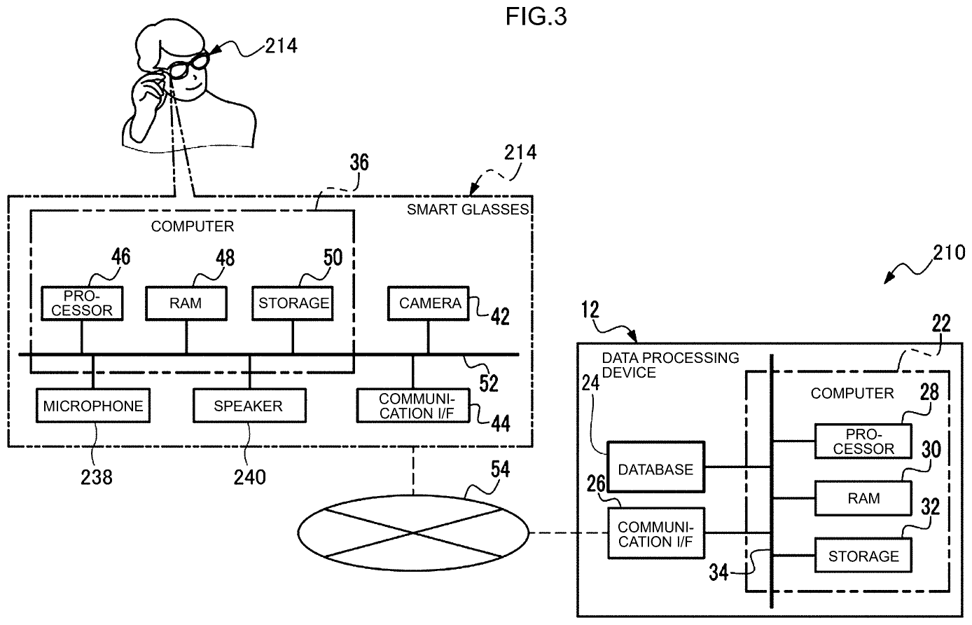

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

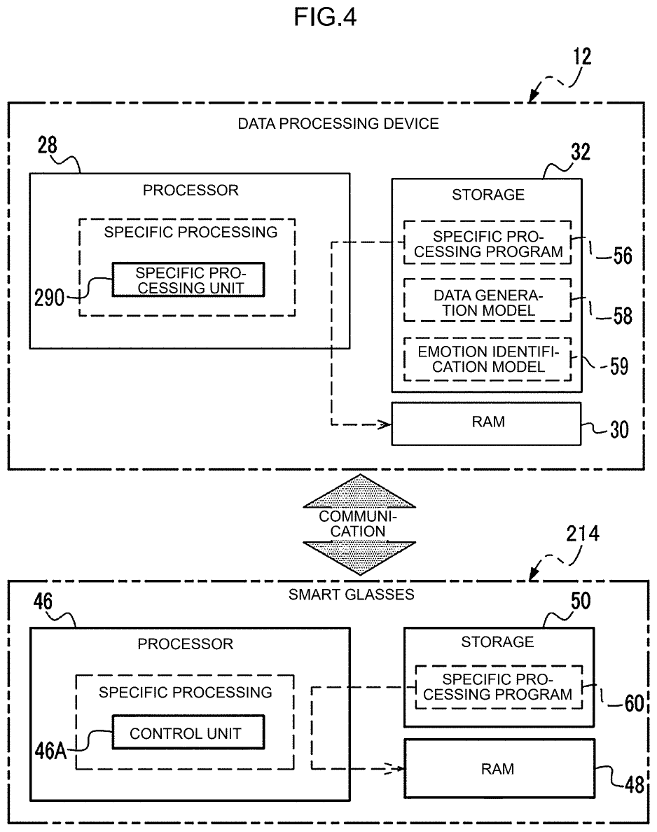

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the above-described collection unit, analysis unit, proposal unit, ordering unit, and recommendation unit is implemented by at least one of, for example, smart glasses 214 and a data processing apparatus 12. For example, the collection unit collects health and exercise information using a camera 42 or a microphone 238 of the smart glasses 214, and collects data by a control unit 46A. The analysis unit is implemented, for example, by a specific processing unit 290 of the data processing apparatus 12 and analyzes the collected data. The proposal unit is implemented, for example, by the specific processing unit 290 of the data processing apparatus 12 and proposes cooking recipes based on analysis results. The ordering unit is implemented, for example, by the control unit 46A of the smart glasses 214 and places orders for ingredients based on the proposed recipes. The recommendation unit is implemented, for example, by the control unit 46A of the smart glasses 214 and provides dining-out information based on the proposed recipes. The correspondence between each unit and the device or control unit is not limited to the above examples and various modifications are possible.

### Third Embodiment

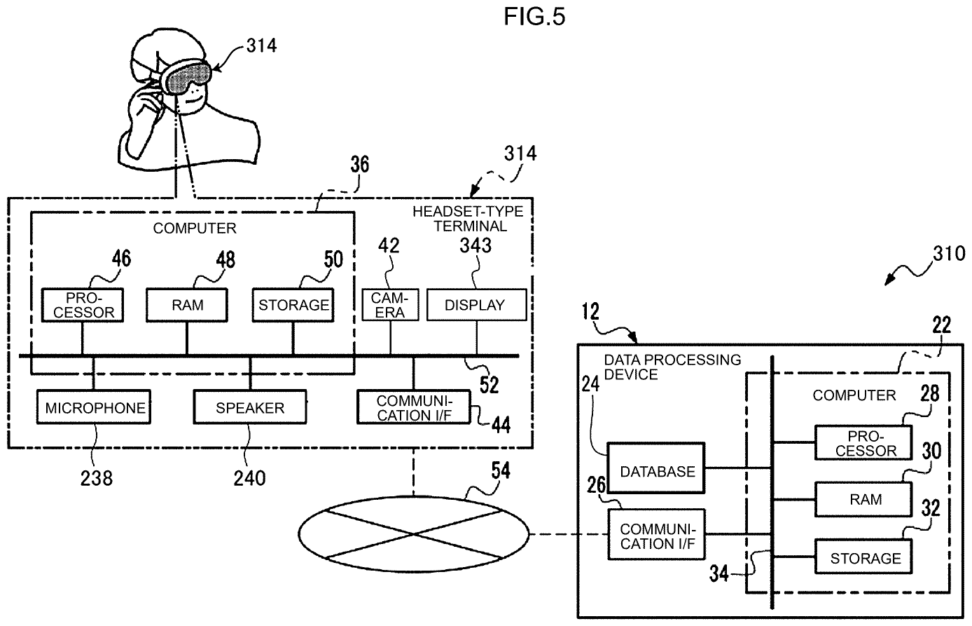

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

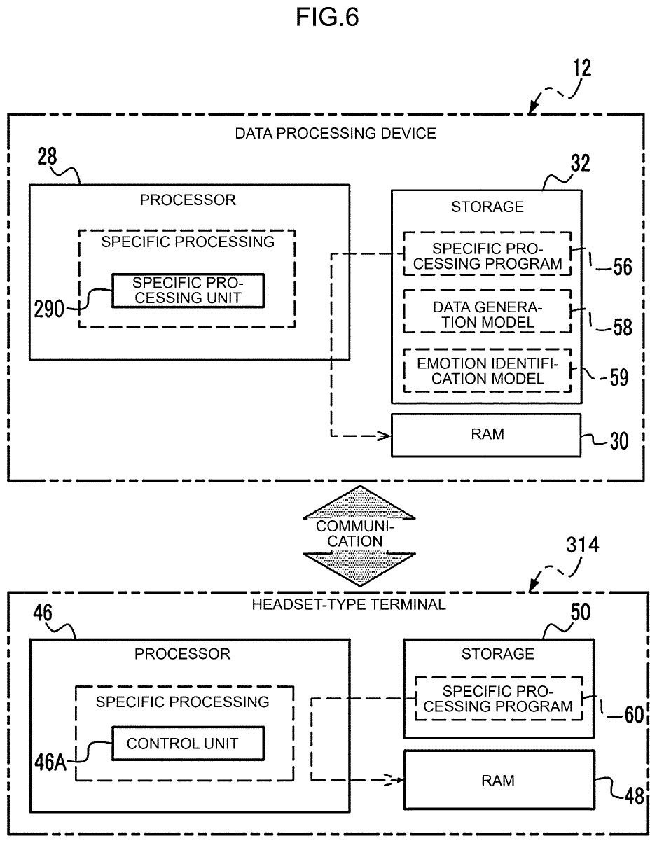

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the above-described collection unit, analysis unit, proposal unit, ordering unit, and recommendation unit is implemented by at least one of, for example, a headset-type terminal 314 and a data processing apparatus 12. For example, the collection unit collects health and exercise information using a camera 42 or a microphone 238 of the headset-type terminal 314, and collects data by a control unit 46A. The analysis unit is implemented, for example, by a specific processing unit 290 of the data processing apparatus 12 and analyzes the collected data. The proposal unit is implemented, for example, by the specific processing unit 290 of the data processing apparatus 12 and proposes cooking recipes based on analysis results. The ordering unit is implemented, for example, by the control unit 46A of the headset-type terminal 314 and places orders for ingredients based on the proposed recipes. The recommendation unit is implemented, for example, by the control unit 46A of the headset-type terminal 314 and provides dining-out information based on the proposed recipes. The correspondence between each unit and the device or control unit is not limited to the above examples and various modifications are possible.

### Fourth Embodiment

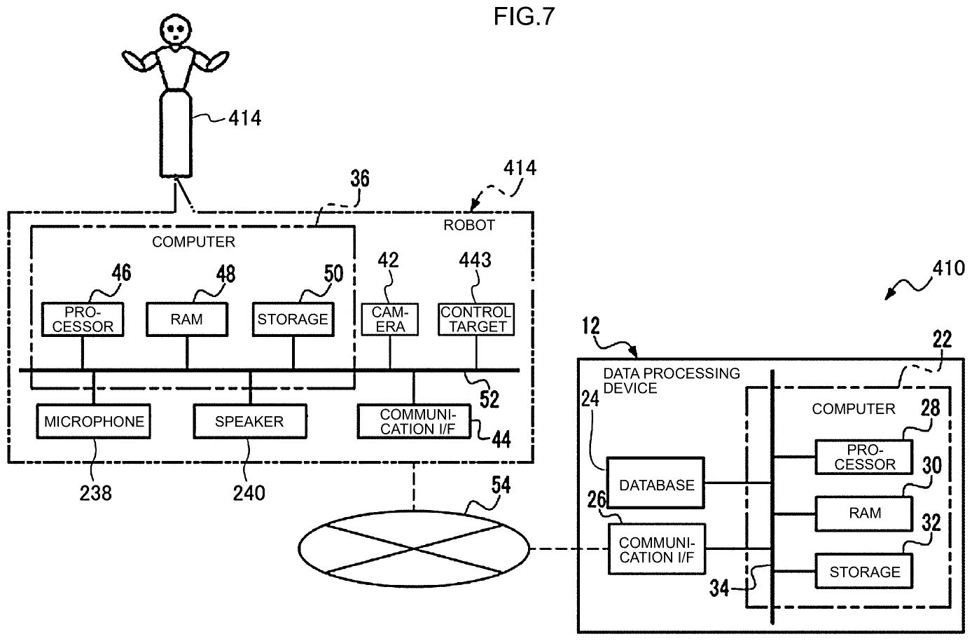

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

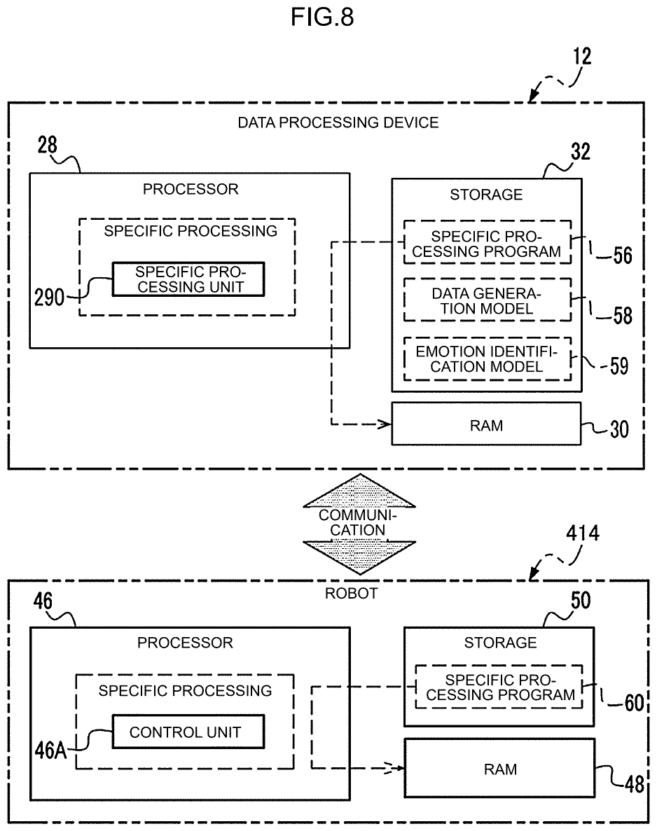

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the above-described collection unit, analysis unit, proposal unit, ordering unit, and recommendation unit is implemented by at least one of, for example, a robot 414 and a data processing apparatus 12. For example, the collection unit collects health and exercise information using a camera 42 or a microphone 238 of the robot 414, and collects data by a control unit 46A. The analysis unit is implemented, for example, by a specific processing unit 290 of the data processing apparatus 12 and analyzes the collected data. The proposal unit is implemented, for example, by the specific processing unit 290 of the data processing apparatus 12 and proposes cooking recipes based on analysis results. The ordering unit is implemented, for example, by the control unit 46A of the robot 414 and places orders for ingredients based on the proposed recipes. The recommendation unit is implemented, for example, by the control unit 46A of the robot 414 and provides dining-out information based on the proposed recipes. The correspondence between each unit and the device or control unit is not limited to the above examples and various modifications are possible.

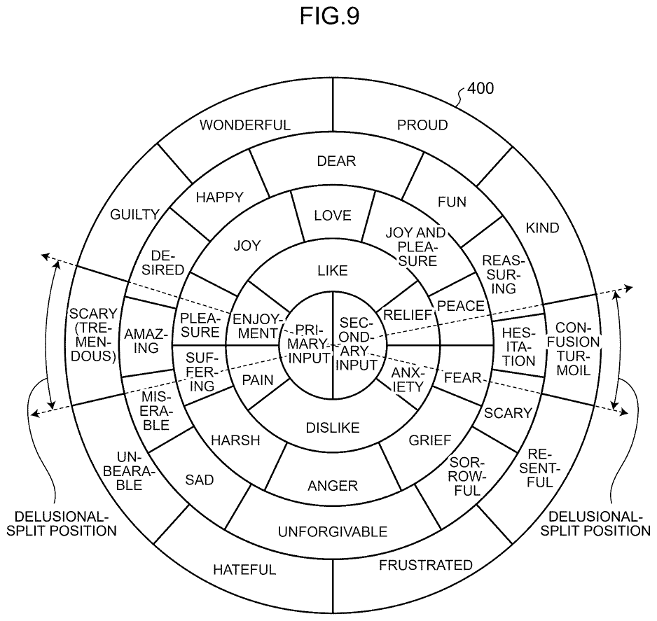

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

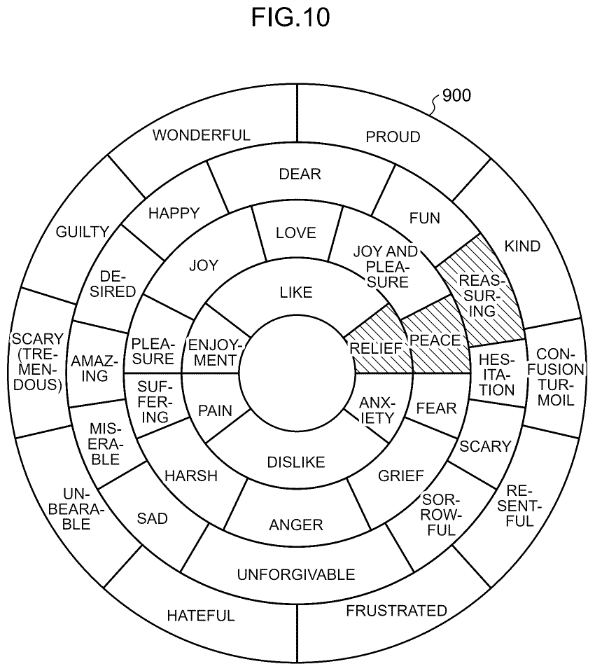

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: a collection unit configured to collect health and exercise information; an analysis unit configured to analyze information collected by the collection unit; a proposal unit configured to propose cooking recipes based on analysis results obtained by the analysis unit; an ordering unit configured to place orders for ingredients based on recipes proposed by the proposal unit; and a recommendation unit configured to provide dining-out information based on recipes proposed by the proposal unit.
(Supplementary Note 2)The system according to Supplementary Note 1, wherein the collection unit is configured to collect health checkup results or daily meal photographs.
(Supplementary Note 3)The system according to Supplementary Note 1, wherein the analysis unit is configured to analyze collected data and grasp health conditions, exercise status, and dietary trends.
(Supplementary Note 4)The system according to Supplementary Note 1, wherein the proposal unit is configured to propose low-calorie cooking recipes for people on a diet.
(Supplementary Note 5)The system according to Supplementary Note 1, wherein the proposal unit is configured to propose nutritionally balanced cooking recipes for people undergoing medical treatment.
(Supplementary Note 6)The system according to Supplementary Note 1, wherein the proposal unit is configured to propose high-protein cooking recipes for professional athletes.
(Supplementary Note 7)The system according to Supplementary Note 1, wherein the ordering unit is configured to automatically select necessary ingredients based on the proposed cooking recipes and place orders with nearby online supermarkets.
(Supplementary Note 8)The system according to Supplementary Note 1, wherein the recommendation unit is configured to search menus of nearby restaurants and cafes based on the proposed cooking recipes, select dining-out information with menus similar to the relevant recipes, and provide recommendations.
(Supplementary Note 9)The system according to Supplementary Note 1, wherein the collection unit is configured to estimate a user's emotions and adjust the timing of collecting health and exercise information based on the estimated emotions.
(Supplementary Note 10)The system according to Supplementary Note 1, wherein the collection unit is configured to analyze a user's past health checkup results and select an optimal collection method.
(Supplementary Note 11)The system according to Supplementary Note 1, wherein the collection unit is configured to perform filtering based on a user's current living conditions and areas of interest when collecting health and exercise information.
(Supplementary Note 12)The system according to Supplementary Note 1, wherein the collection unit is configured to estimate a user's emotions and determine the priority of health and exercise information to be collected based on the estimated emotions.
(Supplementary Note 13)The system according to Supplementary Note 1, wherein the collection unit is configured to prioritize the collection of highly relevant information by considering a user's geographic location when collecting health and exercise information.
(Supplementary Note 14)The system according to Supplementary Note 1, wherein the collection unit is configured to analyze a user's social media activity and collect relevant information when collecting health and exercise information.
(Supplementary Note 15)The system according to Supplementary Note 1, wherein the analysis unit is configured to estimate a user's emotions and adjust the manner of presenting analysis results based on the estimated emotions.
(Supplementary Note 16)The system according to Supplementary Note 1, wherein the analysis unit is configured to adjust the level of detail of analysis based on the importance of health and exercise information during analysis.
(Supplementary Note 17)The system according to Supplementary Note 1, wherein the analysis unit is configured to apply different analysis algorithms according to categories of health and exercise information during analysis.
(Supplementary Note 18)The system according to Supplementary Note 1, wherein the analysis unit is configured to estimate a user's emotions and adjust the length of analysis based on the estimated emotions.
(Supplementary Note 19)The system according to Supplementary Note 1, wherein the analysis unit is configured to determine the priority of analysis based on the timing of collection of health and exercise information during analysis.
(Supplementary Note 20)The system according to Supplementary Note 1, wherein the analysis unit is configured to adjust the order of analysis based on the relevance of health and exercise information during analysis.
(Supplementary Note 21)The system according to Supplementary Note 1, wherein the proposal unit is configured to estimate a user's emotions and adjust the manner of presenting proposals based on the estimated emotions.
(Supplementary Note 22)The system according to Supplementary Note 1, wherein the proposal unit is configured to adjust the level of detail of proposals based on the importance of cooking recipes during proposal.
(Supplementary Note 23)The system according to Supplementary Note 1, wherein the proposal unit is configured to apply different proposal algorithms according to categories of cooking recipes during proposal.
(Supplementary Note 24)The system according to Supplementary Note 1, wherein the proposal unit is configured to estimate a user's emotions and adjust the length of proposals based on the estimated emotions.
(Supplementary Note 25)The system according to Supplementary Note 1, wherein the proposal unit is configured to determine the priority of proposals based on the submission timing of cooking recipes during proposal.
(Supplementary Note 26)The system according to Supplementary Note 1, wherein the proposal unit is configured to adjust the order of proposals based on the relevance of cooking recipes during proposal.
(Supplementary Note 27)The system according to Supplementary Note 1, wherein the ordering unit is configured to estimate a user's emotions and adjust the method of ordering ingredients based on the estimated emotions.
(Supplementary Note 28)The system according to Supplementary Note 1, wherein the ordering unit is configured to analyze a user's past order history and select an optimal ordering method during ordering.
(Supplementary Note 29)The system according to Supplementary Note 1, wherein the ordering unit is configured to customize the means of ordering based on a user's current living conditions during ordering.
(Supplementary Note 30)The system according to Supplementary Note 1, wherein the ordering unit is configured to estimate a user's emotions and determine the priority of orders based on the estimated emotions.
(Supplementary Note 31)The system according to Supplementary Note 1, wherein the ordering unit is configured to select an optimal ordering method by considering a user's geographic location during ordering.
(Supplementary Note 32)The system according to Supplementary Note 1, wherein the ordering unit is configured to analyze a user's social media activity and propose means of ordering during ordering.
(Supplementary Note 33)The system according to Supplementary Note 1, wherein the recommendation unit is configured to estimate a user's emotions and adjust the manner of presenting dining-out information based on the estimated emotions.
(Supplementary Note 34)The system according to Supplementary Note 1, wherein the recommendation unit is configured to refer to a user's past dining-out history and provide optimal recommendations when presenting dining-out information.
(Supplementary Note 35)The system according to Supplementary Note 1, wherein the recommendation unit is configured to estimate a user's emotions and determine the priority of dining-out information based on the estimated emotions.
(Supplementary Note 36)The system according to Supplementary Note 1, wherein the recommendation unit is configured to provide optimal recommendations by considering a user's geographic location when presenting dining-out information.

What is claimed is:

1. A system comprising:
circuitry configured to:
receive, via a communication interface coupled to a packet-switched network, sensor data and image data from a client terminal;
generate feature vectors by performing normalization, missing value imputation, and image feature extraction on the sensor data and the image data;
generate, by inputting the feature vectors into a data generation model obtained by deep learning on a neural network, classification data and inference data, the classification data comprising a status classification and a trend classification;
generate, based on the inference data, a procurement data set by selecting items from a database according to the classification data; and
transmit the inference data and the procurement data set to the client terminal via the communication interface.

2. The system according to claim 1, wherein the sensor data comprises health data and exercise data collected from a wearable device, the health data comprising at least one of heart rate, blood pressure, blood glucose level, or cholesterol level, and the exercise data comprising at least one of step count, calories burned, or sleep duration.

3. The system according to claim 1, wherein the image data comprises meal photograph data in RGB format, and wherein the image feature extraction comprises encoding the meal photograph data using a convolutional neural network to generate image feature vectors.

4. The system according to claim 1, wherein the circuitry is further configured to classify the status classification into one of a plurality of predefined categories and cluster the trend classification into one of a plurality of predefined trend categories by applying a Transformer-based multilayer neural network to the feature vectors.

5. The system according to claim 1, wherein the inference data comprises recipe data specifying an ingredient list, preparation procedures, and nutrient information tailored to the status classification and the trend classification.

6. The system according to claim 5, wherein the circuitry is further configured to apply a calorie restriction algorithm that prioritizes recipe data having less than a predetermined calorie threshold per serving when the status classification indicates a weight reduction category.

7. The system according to claim 5, wherein the circuitry is further configured to apply a nutritional balance algorithm that prioritizes recipe data having a protein-fat-carbohydrate ratio within a predetermined range when the status classification indicates a medical treatment category.

8. The system according to claim 5, wherein the circuitry is further configured to apply a high-protein algorithm that prioritizes recipe data having at least a predetermined protein amount per serving when the classification data indicates a high-intensity exercise status.

9. The system according to claim 1, wherein the circuitry is further configured to generate the procurement data set by referencing inventory status and user preference data stored in the database, excluding items indicated as in-stock in the inventory status, and selecting a fulfillment source based on price, delivery time, and availability.

10. The system according to claim 1, wherein the circuitry is further configured to generate recommendation data by computing a similarity metric between the inference data and entries in a venue information database, filtering the entries based on geographic proximity to a location of the client terminal, and transmitting the recommendation data to the client terminal.

11. The system according to claim 1, wherein the circuitry is further configured to estimate an emotion state of a user by applying an emotion identification model to at least one of audio data, facial image data, or text data received from the client terminal, and adjust a timing of receiving the sensor data based on the estimated emotion state.

12. The system according to claim 11, wherein the circuitry is further configured to adjust an output format of the inference data based on the estimated emotion state by selecting from a plurality of output templates according to an emotion score exceeding a predetermined threshold.

13. The system according to claim 1, wherein the circuitry is further configured to perform time-series analysis on historical sensor data stored in the database, extract indicators having a risk score exceeding a predetermined threshold, and adjust a collection frequency for the sensor data based on the extracted indicators.

14. The system according to claim 1, wherein the circuitry is further configured to receive user profile data from the client terminal, calculate priority scores for categories of the sensor data based on the user profile data, and selectively receive the sensor data according to the priority scores.

15. The system according to claim 1, wherein the circuitry is further configured to receive geographic location data from the client terminal, link the geographic location data with a geographic information database, and adjust a reception priority of the sensor data based on region-specific parameters associated with the geographic location data.

16. The system according to claim 1, wherein the circuitry is further configured to acquire, via the communication interface, social media post data comprising at least one of text data, image data, or activity record data, apply a text analysis model and an image analysis model to the social media post data, and extract supplemental feature vectors for use in generating the classification data.

17. The system according to claim 1, wherein the circuitry is further configured to apply different analysis algorithms to different categories of the sensor data, comprising applying a recurrent neural network to time-series sensor data and applying a convolutional neural network to the image data.

18. A system comprising:
a communication interface configured to communicate with a client terminal via a packet-switched network, the client terminal comprising at least one of a smart device, smart glasses, a headset-type terminal, or a robot;
a processor;
a random-access memory;
a memory storing a data generation model obtained by deep learning on a neural network and an emotion identification model;
a database; and
circuitry configured to:
receive, via the communication interface, sensor data comprising health data and exercise data from a wearable device coupled to the client terminal, and image data comprising meal photograph data captured by a camera of the client terminal;
generate feature vectors by performing normalization, missing value imputation, time-series alignment, and image feature extraction using a convolutional neural network on the sensor data and the image data;
generate, by inputting the feature vectors into the data generation model, classification data comprising a health condition classification, an exercise status estimation, and a dietary trend classification;
generate inference data comprising recipe data specifying an ingredient list, preparation procedures, and nutrient information based on the classification data;
generate a procurement data set by selecting ingredients from the database based on the recipe data, user preference data, and inventory status;
generate recommendation data by computing a similarity metric between the recipe data and venue menu entries stored in the database, filtering by geographic proximity to a location of the client terminal;
estimate an emotion state of a user by applying the emotion identification model to at least one of audio data, facial image data, or text data received from the client terminal; and
transmit the inference data, the procurement data set, and the recommendation data to the client terminal via the communication interface, wherein the processor causes at least one of a display or a speaker of the client terminal to output the transmitted data.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system, the method comprising:
receiving, via a communication interface coupled to a packet-switched network, sensor data and image data from a client terminal;
generating feature vectors by performing normalization, missing value imputation, and image feature extraction on the sensor data and the image data;
generating, by inputting the feature vectors into a data generation model obtained by deep learning on a neural network, classification data and inference data, the classification data comprising a status classification and a trend classification;
generating, based on the inference data, a procurement data set by selecting items from a database according to the classification data; and
transmitting the inference data and the procurement data set to the client terminal via the communication interface.
